(19) United States
(12) Patent Application Publication
(10) Pub. No.: US 2019/0329189 A1
(43) Pub. Date: Oct. 31, 2019

(54) ANGSTROM-SCALE SEPARATIONS BY DESIGN USING PRECISION BIOMIMETIC MEMBRANE

(71) Applicant: The Penn State Research Foundation, University Park, PA (US)

(72) Inventors: Costas D. Maranas, University Park, PA (US); Manish Kumar, State College, PA (US); Ratul Chowdhury, State College, PA (US); Tingwei Ren, State College, PA (US)

(21) Appl. No.: 16/397,416

(22) Filed: Apr. 29, 2019

Related U.S. Application Data
(60) Provisional application No. 62/664,732, filed on Apr. 30, 2018.

(51) Int. Cl. B01D 71/74; B01D 69/02; B01D 61/44; C02F 1/469; G01N 27/447; A61B 5/055; C12Q 1/6869

(52) U.S. Cl. CPC B01D 71/74; B01D 69/02; B01D 61/44; C02F 1/4693; C02F 2103/08; A61B 5/055; C12Q 1/6869; B01D 2325/02; B01D 2325/38; G01N 27/44791

(57) ABSTRACT

The present disclosure describes biomimetic membrane compositions with specifically designed angstrom-scale pores, particularly the continuum of pore sizes between 3 and 10 Å. Further provided are methods of redesigning a protein pore for a desired pore size. The redesigned protein pores and biomimetic membranes based on these proteins provide high selectivity while maintaining high permeability and have many advantages over existing aquaporin-based membranes.

### CROSS REFERENCE TO RELATED APPLICATIONS

This application claims priority to provisional application U.S. Ser. No. 62/664,732, filed Apr. 30, 2018, which is incorporated herein by reference in its entirety.

### TECHNICAL FIELD

The invention relates to membranes with angstrom-scale pores and channels and methods of making and using the same.

### BACKGROUND

Precise chemical separations such as desalination and distillation are among the most challenging and resource-intensive industrial process operations practiced today with an annual energy consumption of ˜50 Quads (50×10¹⁵ BTUs) in the United States alone. Membranes are generally defined as thin, selective barriers that ideally only allow select molecules to permeate through while rejecting others. Permeability and selectivity are key metrics of performance for membrane separations. For biological membranes a nondimensional driving force, defined as the osmotic gradient times the molar volume of water, is used in defining permeability as the fluid flux per unit driving force. Membrane selectivity is consequently defined as the ratio between the permeability of two solutes, two solvents or between a solute and a solvent. Membrane separation offers advantages such as higher selectivity, simpler operation, and higher compactness over other (in many cases thermally driven) separation processes. They are increasingly being applied to a number of industrial sectors including water treatment industrial gas separations, CO₂ capture, food processing, and biopharmaceutical separations. A variety of materials such as synthetic polymers, ceramics, metals, and cellulose can be used to synthesize membranes. However, several challenges still remain in membrane materials design, particularly at the pore scale and translation of such designs to large areas necessary for application. These challenges include: (1) overcoming the trade-off between selectivity and permeability to develop membranes with high selectivity and permeability because improvements in selectivity would simplify multiple separation steps and decrease separation costs significantly; (2) designing angstrom-scale pores that result in the same angstrom-scale separations in synthesized membranes. This would be critical for the efficient separation of small molecules such as ions, gases, and small organics; and (3) synthesizing membranes with uniform pore size distributions. The elimination of polydispersity in membrane pore size would greatly enhance selectivity performance.

To meet these criteria, in recent years, new materials including zeolites, carbon nanotubes, graphene oxide, and membrane proteins, and membrane protein mimics have emerged as advanced membrane materials to assemble the desired pore geometry. However, designing sub-nm pores with perfectly monodispersed distributions is still an unmet challenge and no procedure exists to rationally design the continuum of pore sizes between 3 and 10 Å. Membrane protein channels and biomimetic membranes based on these proteins provide the possibility of realizing sub-nm pore size membranes with perfectly monodisperse pore size distributions, retaining high selectivity while maintaining high permeability. A well-known example of such membranes uses water transport membrane proteins, aquaporins (AQPs), incorporated into liposomes and further stabilized in polyamide polymer membranes for water desalination.

AQPs are well-studied tetrameric water channel proteins present in microbes, plants, and animals. In mammalian cells, fourteen isoforms have been identified with distribution in a wide range of cell types. Each monomer is comprised of eight alpha helices. Six alpha helices are transmembrane in nature and two only partially span the membrane (helices 3 and 7). The pore that results from folding of this protein has an hour-glass structure with a constriction diameter of ˜2.7 Å. The high rate of selective water permeation through AQPs at ˜3 billion water molecules/channel/s, makes them ideal for desalination membranes because any solute of sizes greater than a water molecule is rejected by it. However, the advantages of AQPs for high permeability and high selectivity membrane applications are still being debated due to questions regarding the long-term stability of this alpha helical protein, the low density of proteins embedded in membranes, and its inner pore wall chemistry which forms hydrogen bonds with the permeating water molecules that may impede single channel osmotic permeability. Further, AQP-based membranes do not allow for selective removal of larger solutes in the sub-nm range.

Thus, an objective of the present disclosure is to address the limitations of AQP-based biomimetic membranes and to further enhance the promise of pore-based membranes.

### BRIEF SUMMARY OF PREFERRED EMBODIMENTS

A preferred embodiment is a biomimetic membrane comprising mutant protein pores having an altered pore size and/or altered pore hydrophobicity. In some embodiments, the pore has a size from about 3 Å to about 10 Å. According to the invention, three distinct pore topologies are identified, an off-center pore closure design (OCD), a uniform pore closure design (UCD), and a cork-screw design (CSD).

Preferably, the mutant protein pores include one or more amino acid substitutions in the pore constriction residues relative to the wild type protein sequence. In some embodiments, the wild-type pore-constriction residues are substituted with a hydrophobic amino acid. In some embodiments, the hydrophobic amino acid is a long side-chain, hydrophobic amino acid including tryptophan, phenylalanine, or tyrosine.

Preferably, the protein pore is a β-barrel protein pore. In some embodiments, the β-barrel protein pore is a porin such those present in the outer membrane of gram-negative bacteria. In an exemplary embodiment, the protein pore is Outer Membrane Protein F (OmpF) from E. coli. In yet another embodiment, the protein pore is CsgG.

Preferably, the mutant protein pores are incorporated into biomimetic membranes comprising a lipid or a block copolymer. In an aspect of the invention, compositions comprising the biomimetic membranes are useful in precision separations, air purification, water filtration, and desalination. In another embodiment, the compositions are useful in non-invasive MRI. In yet another embodiment, the compositions are useful in DNA sequencing.

A preferred embodiment includes methods of redesigning the pore size of a protein pore are provided. The methods comprise selecting a desired pore size and altering one or more pore constriction residues relative to the wild type protein sequence. In some embodiments, the desired pore size is from about 3 Å to about 10 Å. In some embodiments, the wild type pore constriction residues are substituted with a hydrophobic amino acid including tryptophan, phenylalanine, or tyrosine. In some embodiments, the replacing results in an off-center pore closure design, a uniform pore closure design, or a cork-screw design. Preferably, membrane contacting residues are also altered to enhance compatible with various membrane materials.

Preferably, the methods comprise incorporating the redesigned protein pore into a membrane. Preferably, the membrane may be a lipid membrane or a synthetic polymer membrane such as block copolymers.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIGS. 1A-B show water wire from Aquaporin 1 (AQP1) used as a template to redesign OmpF pore geometry. In FIG. 1A, the left panel shows a frame from an MD simulation of single-file water permeation through AQP1. The water wire is isolated with its geometry preserved and is thereafter placed in the OmpF pore. The pore-constricting residues are altered such that they fill up the space around the water wire forming a molecular mold of the selective internal geometry of AQP1 within OmpF beta scaffold.

FIG. 1B shows the three distinct internal pore geometries of OmpF that resulted from the employed redesign procedure: (i) off-center pore closure design (OCD), (ii) uniform pore closure design (UCD), and (iii) cork-screw design (CSD).

FIG. 2 shows that the OCD-TFTrp design exhibits steric clash but UCD does not. The OCD_TFTrp design has adjacent tryptophans clashing (left) resulting in some of the side chains facing away from the pore lumen, thereby yielding pore sizes larger than expected. However, in a UCD design (right), an R82L mutation alleviates a steric clash with Trp62 (unlike OCD-TFTrp). UCD designs are seen to intersperse smaller side-chain hydrophobic amino acids between longer ones so their side chains face the pore lumen resulting in smaller pore sizes.

FIGS. 3A-B show twenty OmpF mutants spanning the entire sub-nm range were designed. FIG. 3A is a plot of the number of mutations vs. pore diameter for 20 mutants (including three mutants that were validated experimentally after MD simulations). The general trend indicates that the smaller the desired pore, the greater the number of mutations required. FIG. 3B is a plot of the number of designs for each pore size and type classification.

FIGS. 4A-E show osmotic shock stopped-flow light scattering experiments used to assess transport properties. Stopped-flow light scattering experiments revealed an order of magnitude or higher permeability than aquaporins for WT OmpF protein and its mutants as well as solute retention trends seen in OmpF protein mutants. FIG. 4A shows when OmpF (or OmpF mutant) containing proteoliposomes are mixed with hypertonic solutions, two different transport models can be observed based on whether the solute is permeable to the porin or not. FIG. 4B shows in the stopped-flow setup, for solute excluded model, normalized light scattering intensity levels off during the second stage as there is no inflow of water and solutes; for solute permeable model, normalized light scattering intensity decreased during the second stage due to inflow of water and solutes. FIG. 4C shows OmpF (WT) rejects PEG600 (600 Da) and larger molecules and thus only the PEG600 curves show no decreasing portion of the curve. FIG. 4D shows UCD rejects NaCl (58.5 Da) and larger molecules as there is no decreasing portion of the stopped-flow curve for any of the solutes tested. Curves shown in FIGS. 4C and 4D are averages of 6-10 traces from each stopped-flow light scattering experiment. Each experiment was conducted at least three times with independent vesicle preparations. FIG. 4E is a summary of the estimated solute rejection (light bars) and single-channel permeability (dark bars) of OmpF WT and the three OmpF mutants. The two y-axes represent permeability (left y-axis) or the molecular weight cutoff data (right y-axis). Complete data is shown in FIG. 5.

FIGS. 5A-D show the complete stopped flow results for OmpF wild type and mutant solute rejection: wild type OmpF (WT) showed rejection of PEG600, OCD design OmpF mutant showed rejection of sucrose, CSD design OmpF mutant showed rejection of glucose and UCD design OmpF mutant showed rejection of NaCl. The average vesicle diameter (D in nm) have been shown in each panel. Overall, the average size diameter in the stopped flow experiments is 175 (±13) nm.

FIG. 6 shows net permeability of OmpF wild type and its mutants reconstituted in biomimetic membranes.

FIGS. 7A-B show application of Fluorescence Correlation Spectroscopy (FCS) to determine the number of proteins per vesicle (Nₚᵣₒ/Nᵥₑₛ) FIG. 7A shows addition of detergent to break down labeled protein reconstituted vesicles into micelles. FIG. 7B shows autocorrelation function of vesicles and micelles (from FCS measurements).

FIGS. 8A-F show that MD simulations of OmpF corroborate experimentally observed permeability and selectivity trends. FIG. 8A shows typical simulation system. (Top) Cut-away view of the system revealing a transmembrane water passage through an OmpF monomer. The OmpF monomer, the lipid-bilayer, water molecules, and Na⁺ and Cl⁻ ions are depicted. (Bottom) Top-down view of the system. The OmpF trimer is drawn using a cartoon representation and the lipid-bilayer is shown; water and ions are not shown. FIG. 8B shows simulated osmotic permeability (averaged over 12,500 frames) of OmpF variants and the corresponding experimental values. FIG. 8C shows ionic conductance of OmpF trimers obtained from applied field simulations under a 500 mV transmembrane voltage and averaged over 10,417 frames. FIG. 8D shows water occupancy of OmpF variants. The average location of water molecules in each channel characterized as a 0.3 g/cm³ isosurface of water oxygen density is depicted. For reference, each channel is shown using a semitransparent cartoon representation. FIG. 8E shows major axis dimensions of the pores measured from PoreDesigner before MD and from the last 100 frames of MD from the equilibrated portion of the trajectory. The error bars represent standard deviations about the mean value depicted by the bar height. A 0.4 nm line represents the PoreDesigner design constraint of identifying pore designs smaller than 0.4 nm. FIG. 8F shows the average number of hydrogen bonds made between water and an OmpF monomer in each of the regions depicted in FIG. 8D and averaged over 14,583 frames

FIG. 9 shows sequence alignments of wild type OmpF and the experimentally tested mutants. Segments below represent regions conserved in all four proteins and gaps represent a mutation in that amino acid position in at least one mutant.

FIG. 10 shows a scree plot of the maximum intra-cluster variance against the value k. Here k is the number of clusters. Each cluster contains geometrically similar water wires. Each water wire is represented by a point in the 3D space with coordinates (R1, R2, and C). Two such proximal points are assigned membership to the same cluster. The plot reveals that there are four unique geometries that water assumes while permeating through AQP1.

FIGS. 11A-B illustrate the cascade of steps used for pore profile analysis using a standalone PoreAnalyzer module of PoreDesigner. FIG. 11A shows the 8 Å constriction region is divided into slices every 0.5 Å. Pore area is calculated at each slice and the lowest of them determines the redesigned pore constriction area. FIG. 11B shows the OmpF pore profile generated using PoreAnalyzer module and visualized using PyMOL. The twenty-five pore constriction residues are highlighted. An expanding ellipsoid starting from the pore center coordinate for each slice is fit which just touches the atoms that constitute the pore periphery in that slice. The major and minor axes dimensions of the ellipse thus fit, represents the pore dimensions in that slice. This is repeated for all the slices to obtain the detailed pore geometry along with the pore bottleneck dimensions. A schematic hour-glass shaped internal pore geometry has been overlaid.

FIG. 12 is a schematic representation of the membrane-facing residues which are systematically altered using PoreDesigner to enhance the compatibility of the protein channel with the membrane.

FIG. 13 shows the mechanism by which the presence of a hydrophilic patch prevents proton permeation through redesigned OmpF channels.

FIG. 14 shows engineering DNA translocation channels for nucleotide sequencing. The CsgG nanopore with the ssDNA placed at the center is shown on the left. A trinucleotide is used to mimic a ssDNA because adding more nucleotides would not add significantly to the molecular interactions as the extra nucleotides would be far from the pore constriction amino acids (Tyr51-Asn55) shown on the right panel. PoreDesigner is used to systematically alter the five amino acids to attain targeted hydrophobicity.

### DETAILED DESCRIPTION

The present disclosure relates to biomimetic membrane compositions comprising mutant protein pores. The membrane compositions and methods of preparing the membrane composition have many advantages over existing AQP-type membranes. For example, the membrane compositions can have specifically designed angstrom-scale pores and pore distribution. The membranes can be used for a variety of highly specific separation techniques including, but not limited to, desalination. The embodiments of this invention are not limited to particular membranes or methods of using membranes, which can vary greatly without departing from the scope of the invention.

The pore size and topology can be specifically designed by leveraging a protein design algorithm IPRO (Iterative Protein Redesign and Optimization suite of programs) followed by the subsequent application of molecular dynamics (MD) simulations and validation using stopped-flow light scattering experiments. The core computational module, which we designed and refer to herein as “PoreDesigner,” restricts the modification of the pore constriction residues to long side-chain and hydrophobic amino acids and identifies an optimal set of rotational isomers (from a rotamer library) for the altered residues that avoid backbone and side-chain clashes using a mixed-integer linear optimization program (MILP). Long side-chain hydrophobic amino acids were selected with the dual objective of obtaining smaller pores with hydrophobic side chains that extend into the pore lumen to provide selectivity while maintaining high osmotic permeability based on the hypothesis that reducing water-pore wall interactions will lead to increased permeability. Designs identified by the MILP problem were retained so long as (1) they minimized water wire to pore wall interactions, or (2) had pore sizes smaller than the desired size, using an interaction energy calculation check and a pore area estimation criterion.

Experimental testing of representative mutants from each category revealed a range of designs that maintain water permeabilities exceeding those of classical aquaporins by more than an order of magnitude at over 10 billion water molecules per channel per second while providing specific pore designs that exclude sucrose and larger solutes (>360 Da), glucose and larger solutes (>180 Da), or salt and larger solutes (>58 Da). PoreDesigner provides the ability to design any specified A pore size (spanning 3-10 Å), pore profile, and chemistry that may be ideal for conducting Angstrom-scale aqueous separations.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

### Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, mass, volume, time, distance, molecular weight, water permeability, and hydrophobicity. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

As used herein, the term “analog” means a molecular derivative of a molecule. The term is synonymous with the terms “structural analog” or “chemical analog.”

The methods and compositions of the present invention may comprise, consist essentially of, or consist of the components and ingredients of the present invention as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

As used herein, the prefix “oligo-” refers to a molecular complex comprised of between two and ten monomeric units. For example, oligosaccharides are comprised of between two and ten monosaccharides. Furthermore, unless otherwise specifically limited, the term “oligo-” shall include all possible isomeric configurations of the molecule, including, but are not limited to isotactic, syndiotactic and random symmetries, and combinations thereof. Furthermore, unless otherwise specifically limited, the term “oligo-” shall include all possible geometrical configurations of the molecule.

The terms “polypeptide,” “peptide,” “protein,” and “amino acid sequence” are used interchangeably to refer to a polymer of amino acid residues. The term also applies to amino acid polymers in which one or more amino acids are chemical analogues or modified derivatives of a corresponding naturally-occurring amino acids.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The terms “water soluble” and “water miscible” as used herein, means that the component is soluble or miscible in water at 25° C. preferably at a concentration of 0.01 wt. %, more preferably at 0.1 wt. %, and most preferably at 1 wt. %.

The term “mutant” with respect to a polypeptide, refers to a polypeptide that differs from a specified wild-type or reference polypeptide in that it includes one or more man-made substitutions, insertions, or deletions of an amino acid. Similarly, the term “mutant” with respect to a polynucleotide, refers to a polynucleotide that differs in nucleotide sequence from a specified wild-type or reference polynucleotide. The identity of the wild-type or reference polypeptide or polynucleotide will be apparent from context.

The terms “wild-type” or “reference” with respect to a polypeptide, refer to a naturally-occurring polypeptide that does not include a man-made substitution, insertion, or deletion at one or more amino acid positions. Similarly, the terms “wild-type” or “reference” with respect to a polynucleotide, refer to a naturally-occurring polynucleotide that does not include a man-made nucleotide change.

Reference to the wild-type polypeptide is understood to include the mature form of the polypeptide. A “mature” polypeptide, or mutant thereof, is one in which a signal sequence is absent, for example, cleaved from an immature form of the polypeptide during or following expression of the polypeptide.

The term “isolated” means that the sequence or protein is at least substantially free from at least one other component with which the sequence or protein is naturally associated in nature and as found in nature. In one aspect, the polypeptide(s) for use in the present invention may be in an isolated form.

The term “purified” means that the sequence or protein is in a relatively pure state—e.g., at least about 51% pure, or at least about 75%, or at least about 80%, or at least about 90% pure, or at least about 95% pure or at least about 98% pure. In one aspect, the polypeptide(s) for use in the present invention may be used in a purified form.

As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

Techniques for determining nucleic acid and amino acid sequence identity are known in the art. Typically, such techniques include determining the nucleotide sequence of the mRNA for a gene and/or determining the amino acid sequence encoded thereby and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: blast.ncbi.nlm.nih.gov. GenBank® is the recognized United States-NIH genetic sequence database, comprising an annotated collection of publicly available DNA sequences, and which further incorporates submissions from the European Molecular Biology Laboratory (EMBL) and the DNA DataBank of Japan (DDBJ), see Nucleic Acids Research, January 2013,v 41(D1) D36-42 for discussion. With respect to sequences described herein, the range of desired degrees of sequence identity is approximately 80% to 100% and any integer value there between. Typically, the percent identities between sequences are at least 70%, preferably 80%, more preferably 90%, even more preferably 92%, still more preferably 95%, and most preferably 98% sequence identity.

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein, for instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton (1984) Proteins W.H. Freeman and Company.

### Membrane Proteins

Membrane transport proteins suitable for the present invention are for instance selected from, but not limited to, any membrane protein found in the Transporter Classification Database (TCDB; www.tcdb.org). The TCDB is an International Union of Biochemistry and Molecular Biology (IUBMB)-approved classification system for membrane transport proteins. Preferably, the membrane transport protein comprises a pore. More preferably, the pore protein is a β-barrel-type pore protein. The embodiments of this invention are not limited to particular protein pore, which can vary and are understood by skilled artisans.

Preferably, the pore protein is a β-barrel pore protein. Suitable β-barrel pore proteins include, but are not limited to, outer membrane proteins. In some embodiments, the β-barrel pore protein is a porin. Porins are present in the outer membranes of Gram-negative bacteria, mitochondria and plastids. Examples of porins suitable for the present invention include, but are not limited to, alginate export porin, Anaplasma p44 porin, autotransporter, beta barrel porin, Brucella-Rhizobium porin, Campylobacter jejuni major outer membrane porin, chlamydial porin, corynebacterial porin, Coxiella porin p1, cyanobacterial porin, cyclodextrin porin, electron transport-associated porin, FadL outer membrane protein, fusobacterial outer membrane porin, general bacterial porin, glucose-selective OprB porin, legionella major-outer membrane protein, mitochondrial and plastid porin, mitochondrial protein translocase, mycobacterial porin, nocardial hetero-oligomeric cell wall channel, nucleoside-specific channel-forming outer membrane porin, oligogalacturonate-specific porin, Omp50 porin, OmpA-OmpF porin, OmpG porin, OmpW porin, outer bacterial membrane secretin, outer membrane auxiliary protein, outer membrane beta-barrel endoprotease, outer membrane channel, outer membrane factor, outer membrane fimbrial usher porin, outer membrane lipopolysaccharide export porin, outer membrane porin, outer membrane protein insertion porin, outer membrane receptor, peroxysomal membrane porin, plastid outer envelope porin, poly acetyl glucosamine porin, PorH porin, probable protein translocating Porphyromonas gingivalis porin, proteobacterial outer membrane porin, proteobacterial/verrucomicrobial porin, protochlamydial outer membrane porin, Pseudomonas OprP porin, raffinose porin, Rhodobacter PorCa porin, short chain amide and urea porin, sugar porin, trans-outer membrane electron flow porin, two-partner secretion porin, and δ-proteobacterial porin.

In an exemplary embodiment, the protein pore is outer membrane protein F (OmpF), which is derived from E. coli. OmpF is a member of the general bacterial porin (GBP) family. The general bacterial porin family belongs to the Porin Superfamily I. The wild type OmpF protein structure was obtained from protein database with the accession code, 2OMF. The amino acid sequence of the mature OmpF polypeptide is shown in SEQ ID NO: 1. In some embodiments, the protein pore is a mutant of OmpF selected from the group consisting of SEQ ID NO: 2, SEQ ID NO: 3, and SEQ ID NO: 4.

In some embodiments, one or more pore constriction residues are modified in the OmpF polypeptide of SEQ ID NO: 1. In some embodiments, the mutant OmpF has one or more mutations at the position corresponding to positions 18, 20, 38, 40, 42, 62, 64, 82, 83, 109, 110, 113, 114, 115, 117, 118, 119, 120, 121, 123, 124, 125, 130, 131, 132, and 310 (wherein SEQ ID NO: 1 is used for numbering). In some embodiments, the OmpF mutant has a combination of the mutations and at least 90%, at least 91%, at least 92%, at least 93%, at least 94%, at least 95%, at least 96%, at least 97%, at least 98% or even at least 99% amino acid sequence identity to SEQ ID NO: 1. In some embodiments, the wild type residue is replaced with a tryptophan, phenylalanine, or tyrosine.

In some embodiments, the amino acid substitution is one or more of V18W, L20W, M38W, Y40W, R42W, E62W, E62P, N64W, N64F, R82W, L83W, L109W, G110W, D113W, M114W, L115W, E117W, E117A, F118G, F118W, G119W, G120W, D121W, A123W, Y124W, S125W, V130W, G131F, G131W, R132W, Y310W, and Y310G (wherein SEQ ID NO: 1 is used for numbering).

In some embodiments, the protein pore is Curli production assembly/transport component CsgG. The amino acid sequence of the mature CsgG polypeptide is shown in SEQ ID NO: 5. In some embodiments, one or more residues at the pore mouth are mutated in the CsgG polypeptide of SEQ ID NO: 5. Preferably, the hydrophobicity of the pore mouth of the CsgG protein is altered. In some embodiments, the mutant CsgG has one or more mutations at the position corresponding to positions 51, 52, 53, 54, and 55 (wherein SEQ ID NO: 5 is used for numbering). In some embodiments, the wild type residues are replaced with residues with low, medium, and/or high hydrophobicities. Lysine, arginine, and cystine are low hydrophobicity amino acids, leucine, isoleucine, and valines are medium, and phenylalanine and tryptophans are highly hydrophobic residues. In some embodiments, the CsgG mutant has a combination of the mutations and at least 90%, at least 91%, at least 92%, at least 93%, at least 94%, at least 95%, at least 96%, at least 97%, at least 98% or even at least 99% amino acid sequence identity to SEQ ID NO: 5. The mutant CsgG enhances resolution of the signals from all four types of nucleotides (adenine, thymine, cytosine, and guanine) relative to the wild type CsgG.

PoreDesigner can be applied to tune pore size and geometry for any porin system, particularly those with a known structure. By means of example and not limitation, experimentally (e.g., using X-ray crystallography or NMR spectroscopy) resolved conformations of biomolecules and in particular many peptides, polypeptides, proteins, nucleic acids and complexes are published in scientific literature and compiled in public databases, such as notably the Protein Data Bank database (Berman et al. 2000. Nucleic Acids Res 28(1): 235-242; www.wwpdb.org). Homology models are useful when there is no experimental information available on the protein of interest. Homology modeling (also known as comparative modeling or knowledge-based modeling) methods develop a three-dimensional model from a polypeptide sequence based on the structures of known proteins.

Preferably the protein pores can be designed with specific pore sizes. Preferably the pores have a diameter of less than about 20 Å, 19 Å, 18 Å, 17 Å, 16 Å, 15 Å, 14 Å, 13 Å, 12 Å, 11 Å, 10 Å, 9 Å, 8 Å, 7 Å, 6 Å, 5 Å, or 4 Å. Preferably, the pores have a diameter of at least about 1 Å, 2 Å, 3 Å, 4 Å, 5 Å, 6 Å, 7 Å, 8 Å, 9 Å, or 10 Å. In a preferred embodiment the pores have major and minor axes lengths of between about 7 Å and about 20 Å and 3 Å and about 15 Å, respectively. In an aspect of the invention, the pores can be designed with uniform pore sizes or varying pore sizes.

Preferably, the pores can have a specific solute selectivity including complete salt rejection. The compositions and pores have specific solute selectivity (including, but not limited to, complete salt rejection) and high osmotic permeabilities. The improved solute selectivity and high osmotic permeabilities provide an improvement over aquaporins.

In some embodiments, the pore excludes sucrose and larger solutes (>360 Da). In some embodiments, the pore excludes glucose and larger solutes (>180 Da). In some embodiments, the pore excludes salt and larger solutes (>58 Da). In some embodiments, the pore excludes molecules larger than 40 Da, 50 Da, 60 Da, 70 Da, 80 Da, 90 Da, 100 Da, 110 Da, 120 Da, 130 Da, 140 Da, 150 Da, 160 Da, 170 Da, 180 Da, 190 Da, 200 Da, 210 Da, 220 Da, 230 Da, 240 Da, 250 Da, 260 Da, 270 Da, 280 Da, 290 Da, 300 Da, 310 Da, 320 Da, 330 Da, 340 Da, 350 Da, 360 Da, 370 Da, 380 Da, 390 Da, 400 Da, 410 Da, 420 Da, 430 Da, 440 Da, 450 Da, 460 Da, 470 Da, 480 Da, 490 Da, 500 Da, 510 Da, 520 Da, 530 Da, 540 Da, 550 Da, 560 Da, 570 Da, 580 Da, 590 Da, or 600 Da.

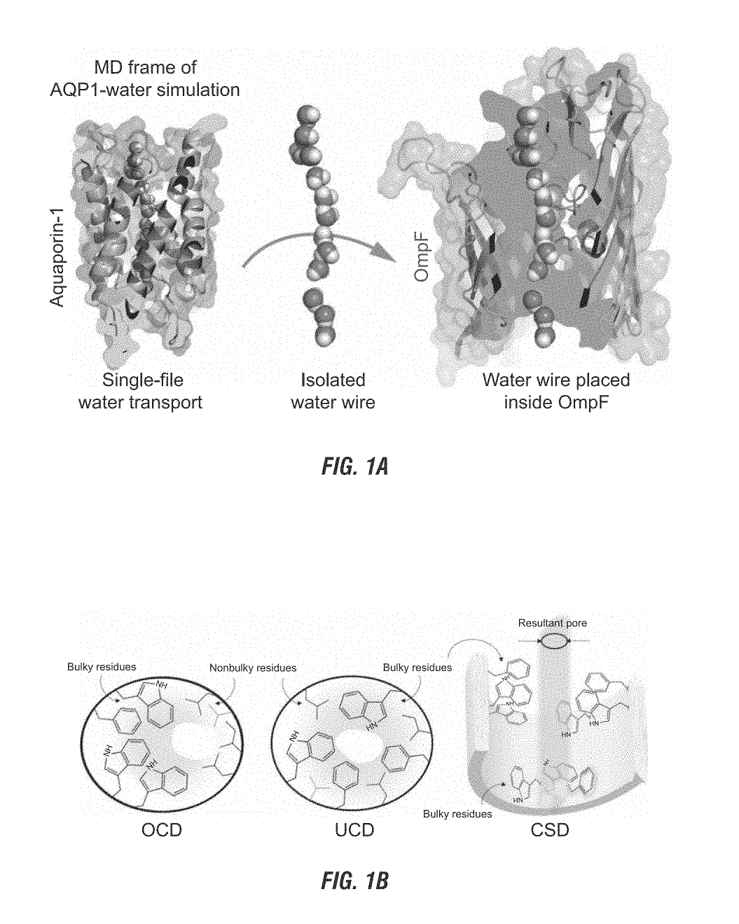

The pores can be designed with a specific pore topology/geometry. Preferably, the pore topology is one of three distinct pore topologies: off-center pore closure, uniform pore closure, and cork-screw design. Off-center pore closure designs (OCD) involve a pore center that is displaced towards the perimeter compared to the wild type protein pore utilizing long side chain hydrophobic resides on one side of the pore and smaller ones on the other. Uniform pore closure designs (UCD) have a smaller but nearly co-centric pore eyelet diameter resulting from an orderly distribution of similar-side chain size hydrophobic amino acids along the pore perimeter. Cork-screw designs (CSD) introduce a lateral twist along the pore axis stemming from alternating stacking long with short side-chain amino acids (as shown in FIG. 1, which provides a non-limiting example of a CSD).

The pore constriction residues of a protein pore can be changed to alter the desired pore size and the pore topology to form an off-center pore closure design (OCD), a uniform pore closure design (UCD), or a cork-screw design (CSD). In some embodiments, the pore constriction residues of wild-type protein pore are replaced with hydrophobic amino acids including tryptophan (Trp), phenylalanine (Phe), tyrosine (Tyr), isoleucine (Ile), methionine (Met), leucine (Leu), proline (Pro), valine (Val), and alanine (Ala). In some embodiments, the pore constriction residues of wild-type protein pore are replaced with short side-chain, hydrophobic amino acids. In some embodiments, the pore constriction residues of wild-type protein pore are replaced with long side-chain, hydrophobic amino acids such as tryptophan, phenylalanine, and tyrosine. In some embodiments, the pore constriction residues of wild-type protein pore are replaced with a mixture of short side-chain, hydrophobic amino acids and long side-chain, hydrophobic amino acids. In some embodiments, a minimum of 50% of the substituted residues are either tryptophan, phenylalanine, or tyrosine.

In some embodiments, one or more of the pore constriction residues of wild type protein pore are replaced with hydrophobic amino acids. In some embodiments, the total number of amino acids substituted can be, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 75, 100 or more amino acids, dependent upon the total number of pore constriction residues of the wild type protein pore.

The membrane-facing residues of the protein can also be altered to make the protein compatible with various membrane materials (e.g., lipid, polymer, etc). In some embodiments, the protein pore is a wild-type protein pore or mutant protein pore. In some embodiments, the composition comprises a mixture of protein pores, including both wild-type protein pores and mutant protein pores.

The protein pores are designed to have enhanced permeability compared to aquaporins. In some embodiments, the water permeability of the protein pore is an order of magnitude higher than of AqpZ. In some embodiments, the water permeability can be, for example, about 1×10⁻¹² cm³/s, 2×10⁻¹² cm³/s, 3×10⁻¹² cm³/s, 4×10⁻¹² cm³/s, 5×10⁻¹² cm³/s, 6×10⁻¹² cm³/s, 7×10⁻¹² cm³/s, 8×10⁻¹² cm³/s, 9×10⁻¹² cm³/s, or more. In some embodiments, the water permeability is at least about 2×10⁻¹² cm³/s, more preferably at least about 4×10⁻¹² cm³/s. In some embodiments, the water permeability is from about 1×10⁻¹² cm³/s to about 9×10⁻¹² cm³/s. In some embodiments, the water permeability is from about 2×10⁻¹² cm³/s to about 6×10⁻¹² cm³/s.

In some embodiments, the protein pore is further modified to reject protons. When water permeates through an aquaporin as a molecule-wide water stream, near the constriction region, a polar amino acid (asparagine) pulls the oxygen of the water towards itself thus breaking the water stream. Beyond this point, downstream through the channel, the water molecules appear rotated by a complete 180 degrees. This prevents the protons from jumping from one water molecule to the next (via intermediate hydronium ion formation) due to a larger jump length. The protein pores of the present invention can be similarly modified to reject proteins. In some embodiments, one or more hydrophilic residues are introduced at the pore constriction in order to reject protons. In a preferred embodiment, one or more asparagine residues are introduced. In an exemplary embodiment, the asparagine residues are introduced at the position corresponding to positions 82 and/or 132 of OmpF (wherein SEQ ID NO: 1 is used for numbering).

### Biomimetic Membranes

The present disclosure relates to biomimetic membranes comprising angstrom-size pores with no polydispersity and embedded in a suitable matrix. The membranes are particularly suitable for precision separations. Thus, the membranes can be used in a variety of separations, biomimetic devices, etc. Precision separations can be performed with the use of the biomimetic membranes in filtration devices, including, but not limited to, masks, air purifiers, water filters, desalination devices, and biomimetic devices. The biomimetic membranes can be used for non-invasive magnetic resonance imaging (MRI) and DNA sequencing. The compositions described herein can provide substantial energy savings in applications ranging from water treatment to small molecule bioseparations. While, monodisperse angstrom-sized pores in the form of membrane proteins are commonplace in biological membranes they have been difficult to implement in synthetic industrial membranes. They have only recently become available as commercial products in the form of aquaporin-based membranes. Unfortunately, in these membranes, improvements in selectivity and permeability have remained modest with no ability to control selectivity. The technology as described in the present disclosure overcomes these problems and difficulties by providing a robust beta-barrel outer membrane protein as a “scaffold” to form angstrom-scale pores.

In one aspect, the present invention provides biomimetic membrane compositions. The term biomimetic membrane includes one or more membranes or walls or shells. The biomimetic membranes include vesicles (e.g. liposomes, micelles, polymerosome, nanoparticles and microbubbles) surrounding an internal void that could be empty or filled, e.g. filled with a gas, liquid or precursor thereto. The biomimetic membranes include planar biomimetic membranes as well as solid-supported membranes such as solid-supported lipid bilayers and tethered lipid bilayers, or membranes comprising vesicles. In some embodiments, the biomimetic membranes comprise one or more lipids and/or one or more polymers. The term lipid includes agents exhibiting amphipathic characteristics causing it to spontaneously adopt an organized structure in water wherein the hydrophobic portion of the molecule is sequestered away from the aqueous phase. In some embodiments, the biomimetic membranes comprise polymerizable lipids. In some embodiments, the biomimetic membranes comprise one or more lipids, at least one of which is polymerizable. In some embodiments, the biomimetic membranes also contain one or more polypeptides, and/or other functional molecules. The biomimetic membranes of the invention may also include any other materials or combination thereof known to those skilled in the art as suitable for biomimetic membrane construction.

Lipids

In one aspect, the biomimetic membranes of the invention comprise one or more lipids. Examples of useful lipids for the formation of lipid membranes (e.g. monolayer, bilayers, planar or vesicles) to be used in the biomimetic membranes of the invention, include but are not limited to:

(i) Phosphatidylcholines: 1,2-dimyristoylphosphatidylcholine (DMPC); 1,2-dipalmitoylphosphatidylcholine (DPPC); 1,2-distearoylphosphatidylcholine (DSPC); 1,2-dioleoylphosphatidylcholine (DOPC); 1,2-dimyristoleoylphosphatidylcholine; 1,2-dipalmitoleoylphosphatidylcholine; 1,2-dipetroselinoylphosphatidylcholine; 1,2-dielaidoylphosphatidylcholine; 1,2-dilinoleoylphosphatidylcholine; 1,2-dilinolenoylphosphatidylcholine; 1,2-dieicosenoylphosphatidylcholine; 1,2-diarachidonoylphosphatidylcholine; 1,2-dierucoylphosphatidylcholine; 1,2-dnervonoylphosphatidylcholine; 1-palmitoyl-2-oleoylphosphatidylcholine (POPC); 1-palmitoyl-2-linoleoylphosphatidylcholine; 1-palmitoyl-2-arachidonoylphosphatidylcholine; 1-palmitoyl-2-docosahexaenoylphosphatidylcholine; 1-stearoyl-2-oleoylphosphatidylcholine (SOPC); 1-stearoyl-2-linoleoylphosphatidylcholine; 1-stearoyl-2-arachidonoylphosphatidylcholine; 1-stearoyl-2-docosahexaenoylphosphatidylcholine; 1-oleoyl-2-palmitoylphosphatidylcholine; 1-oleoyl-2-stearoylphosphatidylcholine; 1,2-didocosahexaenoylphosphatidylcholine;
(ii) Phosphatidylethanolamines: 1,2-dimyristoylphosphatidylethanolamine (DMPE); 1,2-dipalmitoylphosphatidylethanolamine (DPPE); 1,2-distearoylphosphatidylethanolamine (DSPE); 1,2-dioleoylphosphatidylethanolamine (DOPE); 1-palmitoyl-2-oleoylphosphatidylethanolamine (POPE); 1-palmitoyl-2-linoleoylphosphatidylethanolamine; 1-palmitoyl-2-arachidonoylphosphatidylethanolamine; 1-palmitoyl-2-docosahexaenoylphosphatidylethanolamine; 1-stearoyl-2-oleoylphosphatidylethanolamine (SOPE); 1-stearoyl-2-linoleoylphosphatidylethanolamine; 1-stearoyl-2-arachidonoylphosphatidylethanolamine; 1-stearoyl-2-docosahexaenoylphosphatidylethanolamine; 1,2-dielaidoylphosphatidylethanolamine; 1,2-dilinoleoylphosphatidylethanolamine; 1,2-dilinolenoylphosphatidylethanolamine; 1,2-diarachidonoylphosphatidylethanolamine; 1,2-didocosahexaenoylphosphatidylethanolamine; 1,2-dipalmitoleoylphosphatidylethanolamine;
(iii) Phosphatidylglycerols: 1,2-dimyristoylphosphatidylglycerol (DMPG); 1,2-dipalmitoylphosphatidylglycerol (DPPG); 1,2-distearoylphosphatidylglycerol (DSPG); 1,2-dioleoylphosphatidylglycerol (DOPG); 1-palmitoyl-2-oleoylphosphatidylglycerol (POPG); 1-palmitoyl-2-linoleoylphosphatidylglycerol; 1-palmitoyl-2-arachidonoylphosphatidylglycerol; 1-palmitoyl-2-docosahexaenoylphosphatidylglycerol; 1-stearoyl-2-oleoylphosphatidylglycerol (SOPG); 1-stearoyl-2-linoleoylphosphatidylglycerol; 1-stearoyl-2-arachidonoylphosphatidylglycerol; 1-stearoyl-2-docosahexaenoylphosphatidylglycerol;
(iv) Phosphatidylserines: 1-palmitoyl-2-oleoylphosphatidylserine (POPS); 1-palmitoyl-2-linoleoylphosphatidylserine; 1-palmitoyl-2-arachidonoylphosphatidylserine; 1-palmitoyl-2-docosahexaenoylphosphatidylserine; 1-stearoyl-2-oleoylphosphatidylserine (SOPS); 1-stearoyl-2-linoleoylphosphatidylserine; 1-stearoyl-2-arachidonoylphosphatidylserine; 1-stearoyl-2-docosahexaenoylphosphatidylserine; 1,2-dimyristoylphosphatidylserine (DMPS); 1,2-dipalmitoylphosphatidylserine (DPPS); 1,2-distearoylphosphatidylserine (DSPS); 1,2-dioleoylphosphatidylserine (DOPS); 1,2-didocosahexaenoylphosphatidylserine; 1,2-dierucoylphosphatidylserine;
(v) Special lipids: Cardiolipin; Bipolar lipids;
(vi) Polymerizable lipids: 1,2-di-10, 12-tricosadiynoyl-sn-glycero-3-phosphocholine (DTPC); 1,2-di-10, 12-tricosadiynoyl-sn-glycero-3-phosphoethanolamine (DTPE); 1-palmitoyl-2, 10,12-tricosadiynoyl-sn-glycero-3-phosphoethanolamine (PTPE); (DC8,9PC [1,2-bis(10, 12-tricosadiynoyl)-sn-glycero-3-phosphocholine]; diPhyPC [1,2-diphytanoyl-sn-glycero-3-phosphocholine].
(vii) Natural lipid extracts: Egg yolk phosphatidylcholine; Bovine heart phosphatidylcholine; Brain phosphatidylcholine; Bovine liver phosphatidylcholine; Soybean phosphatidylcholine; E. Coli phosphatidylethanolamine; Bovine Heart phosphatidylethanolamine; Brain phosphatidylethanolamine; Bovine Liver phosphatidylethanolamine; Egg phosphatidylethanolamine; Bovine liver phosphatidylinositol; Soybean phosphatidylinositol; Brain phosphatidylserine; Soy phosphatidylserine.

The component lipids of the biomimetic membranes can be purified and characterized individually using standard, known techniques and then combined in controlled fashion to produce the final particle. The biomimetic membranes can be constructed to mimic native cell membranes or present functionality. Additionally, the biomimetic membranes can have a well-defined monolayer or bilayer structure that can be characterized by known physical techniques such as transmission electron microscopy and atomic force microscopy.

Polymers

In one aspect, the biomimetic membranes of the invention comprise one or more polymers. In some embodiments, the biomimetic membranes comprise block copolymer membranes. In some embodiments, the polypeptide(s) described herein are incorporated into the block copolymer membranes.

Various types of amphiphilic copolymers can be used. In one embodiment, the copolymer is an ABA copolymer, where A is hydrophilic and B is hydrophobic where A is the same or different hydrophilic segments and B is a hydrophobic B segment. Thus, the term “ABA copolymer” includes an ABC copolymer, where the hydrophilic segments A and C are different.

The block copolymer includes at least one segment B that includes a hydrophobic polymer. Any of a number of hydrophobic polymers can be used, such as, but not limited to, polysiloxane such as polydimethylsiloxane and polydiphenylsiloxane, perfluoropolyether, polystyrene, polyoxypropylene, polyvinylacetate, polyoxybutylene, polyisoprene, polybutadiene, polyvinylchloride, polyalkylacrylate (PAA), polyalkylmethacrylate, polyacrylonitrile, polypropylene, PTHF, polymethacrylates, polyacrylates, polysulfones, polyvinylethers, and poly(propylene oxide), and copolymers thereof.

In some embodiments, the hydrophobic segment contains a predominant amount of hydrophobic monomers. A hydrophobic monomer is a monomer that typically gives a homopolymer that is insoluble in water and can absorb less than 10% by weight of water.

Suitable hydrophobic monomers are C1-C18 alkyl and C3-C18 cycloalkyl acrylates and methacrylates, C3-C18 alkylacrylamides and -methacrylamides, acrylonitrile, methacrylonitrile, vinyl C1-C18 alkanoates, C2-C18 alkenes, C2-C18 haloalkenes, styrene, (lower alkyl)styrene, C4-C12 alkyl vinyl ethers, C2-C10 perfluoro-alkyl acrylates and methacrylates and correspondingly partially fluorinated acrylates and methacrylates, C3 through C12 perfluoroalkylethylthiocarbonylaminoethyl acrylates and methacrylates, acryloxy- and methacryloxyalkylsiloxanes, N-vinylcarbazole, C1 through C12 alkyl esters of maleic acid, fumaric acid, itaconic acid, mesaconic acid, vinyl acetate, vinyl propionate, vinyl butyrate, vinyl valerate, chloroprene, vinyl chloride, vinylidene chloride, vinyltoluene, vinyl ethyl ether, perfluorohexyl ethylthiocarbonylaminoethyl methacrylate, isobornyl methacrylate, trifluoroethyl methacrylate, hexafluoroisopropyl methacrylate, hexafluorobutyl methacrylate, tristrimethylsilyloxysilylpropyl methacrylate (TRIS), and 3-methacryloxypropylpentamethyldisiloxane.

In some embodiments, the hydrophobic polymer is one which displays a relatively high oxygen diffusion rate there through, such as, but not limited to, polysiloxanes, perfluoroalkyl ethers, specific unsaturated polymers, and polysulfones. In one embodiment, the hydrophobic polymer is a polysiloxane block having terminal alkylene groups.

In some embodiments, the hydrophobic polymer includes a perfluoroalkyl-polyether block. In other embodiments, the hydrophobic polymer includes an unsaturated polymer, such as a polymer of a conjugated aliphatic or alicyclic diene, which may be substituted by halogen or lower alkyl, a polymer of an alkyne or dialkyne, which may be substituted by lower alkyl or trimethylsilyl, a copolymer of a conjugated diene and a hydrophilic or hydrophobic vinylic monomer, and also partially hydrated derivatives of these compounds.

Specific examples of polymers of conjugated dienes are cis-, trans-, iso- or syndiotactic poly-1,2-butadiene, poly-1,4-butadiene or polyisoprene, poly-pentenamer, polychloroprene and polypiperylen. Other examples of copolymers are butadiene- or isoprene-copolymers with hydrophilic or hydrophobic vinylic monomers, such as acrylonitrile, styrene, acrylic acid or hydroxyethylmethacrylate. An example of a polyalkyne is poly-1-trimethylsilyl-propyne. In some embodiments, examples of polymers included unsaturated polymers are syndiotactic poly-1,2-butadiene, poly-1,4-butadiene and polyisoprene. An especially preferred unsaturated polymer is poly-1-trimethylsilyl-propyne. Another especially preferred unsaturated polymer is poly-1,4-butadiene. The hydrophobic polymer may include a single type of polymer or more than one type of polymer, such as two or more of those discussed above. The mean molecular weight of one segment B is in the range from about 500 to about 50,000, preferably in the range from about 800 to about 15,000, more preferably in the range of about 1,000 to 12,000, particularly preferably in the range from about 5,000 to about 12,000.

In some embodiments in addition to the hydrophobic segment B, the amphiphilic segmented copolymer includes at least one segment A which includes at least one hydrophilic polymer, such as, but not limited to, polyoxazoline, polyethylene glycol, polyethylene oxide, polyvinyl alcohol, polyvinylpyrrolidone, polyacrylamide, poly(meth)acrylic acid, polyethylene oxide-co-polypropyleneoxide block copolymers, poly(vinylether), poly(N,N-dimethylacrylamide), polyacrylic acid, polyacyl alkylene imine, polyhydroxyalkylacrylates such as hydroxyethyl methacrylate (HEMA), hydroxyethyl acrylate, and hydroxypropyl acrylate, polyols, and copolymeric mixtures of two or more of the above mentioned polymers, natural polymers such as polysaccharides and polypeptides, and copolymers thereof, and polyionic molecules such as polyallylammonium, polyethyleneimine, polyvinylbenzyltrimethylammonium, polyaniline, sulfonated polyaniline, polypyrrole, and polypyridinium, polythiophene-acetic acids, polystyrenesulfonic acids, zwitterionic molecules, and salts and copolymers thereof.

In some embodiments, the hydrophilic segment preferably contains a predominant amount of hydrophilic monomers. A hydrophilic comonomer is a monomer that typically gives a homopolymer that is soluble in water or can absorb at least 10% by weight of water.

Suitable hydrophilic monomers are hydroxyl-substituted lower alkyl acrylates and methacrylates, acrylamide, methacrylamide, (lower alkyl) acrylamides and methacrylamides, N,N-dialkyl-acrylamides, ethoxylated acrylates and methacrylates, polyethyleneglycol-mono methacrylates and polyethyleneglycolmonomethylether methacrylates, hydroxyl-substituted (lower alkyl)acrylamides and methacrylamides, hydroxyl-substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone, 2-vinyloxazoline, 2-vinyl-4,4′-dialkyloxazolin-5-one, 2- and 4-vinylpyridine, vinylically unsaturated carboxylic acids having a total of 3 to 5 carbon atoms, amino(lower alkyl)-(where the term amino also includes quaternary ammonium), mono(lower alkylamino)(lower alkyl) and di(lower alkylamino)(lower alkyl) acrylates and methacrylates, allyl alcohol, 3-trimethylammonium 2-hydroxypropylmethacrylate chloride (Blemer.QA, for example from Nippon Oil), dimethylaminoethyl methacrylate (DMAEMA), dimethylaminoethylmethacrylamide, glycerol methacrylate, and N-(1,1-dimethyl-3-oxobutyl)acrylamide.

In some embodiments, the segment A includes a polymer displaying a relatively high water or ion diffusion rate there through. Specific examples of hydrophilic monomers from which such polymers can be made are cyclic imino ethers, vinyl ethers, cyclic ethers including epoxides, cyclic unsaturated ethers, N-substituted aziridines, beta-lactones and beta-lactams. Further suitable monomers include ketene acetals, vinyl acetals and phosphoranes. Suitable cyclic imino ethers include 2-oxazoline. If a 2-oxazoline having an alkenyl group in 2 position is used as hydrophilic monomer, a polymerizable unsaturated group is provided within segment A (in a side chain) of the amphiphilic segmented copolymer to serve as the polymerizable unsaturated group necessary for the final polymerization to obtain a polymeric product or as an additional polymerizable unsaturated group which offers the possibility of direct crosslinking in the preparation of the polymer. In some embodiments, the cyclic imino ether is 2-methyloxazoline. The most preferred vinyl ethers are methyl vinyl ether, ethyl vinyl ether and methoxy ethyl vinyl ether.

In some embodiments, the mean molecular weight of one segment A is in the range from about 500 to about 50,000, from about 800 to about 15,000, from about 1,000 to 12,000, particularly from about 5,000 to about 12,000.

It is understood that modifications which do not substantially affect the activity the various embodiments of this invention are also provided within the definition of the invention provided herein. Accordingly, the following examples are intended to illustrate but not limit the present invention.

### EXAMPLES

### Example 1: Dialing in Solute Selectivity in a Robust and Highly Permeable Outer Membrane Protein Pore

In this example, we outline the systematic workflow PoreDesigner for a predictive platform to utilize the OmpF “scaffold” to design angstrom-scale pore sizes with specific solute selectivity and high osmotic permeabilities compared to aquaporins. This was accomplished by leveraging the protein design algorithm IPRO (Iterative Protein Redesign and Optimization suite of programs) followed by the subsequent application of molecular dynamics (MD) simulations and validation using stopped-flow light scattering experiments. The core computational module of PoreDesigner restricts the modification of the pore constriction residues to long side-chain and hydrophobic amino acids and identifies an optimal set of rotational isomers (from a rotamer library) for the altered residues that avoid backbone and side-chain clashes using a mixed-integer linear optimization program (MILP). Long side-chain hydrophobic amino acids were selected with the dual objective of obtaining smaller pores with hydrophobic side chains that extend into the pore lumen to provide selectivity while maintaining high osmotic permeability based on the hypothesis that reducing water-pore wall interactions will lead to increased permeability. Designs identified by the MILP problem were retained so long as (1) they minimized water wire to pore wall interactions, or (2) had pore sizes smaller than the desired size, using an interaction energy calculation check and a pore area estimation criterion. Structural investigation on the designs revealed three distinct topologies of pore geometries: (a) uniform pore closure designs (UCD) with a smaller but nearly co-centric pore eyelet diameter resulting from an orderly distribution of similar-side chain size hydrophobic amino acids along the pore perimeter, (b) off-center pore closure designs (OCD) which involve a pore center that is displaced towards the perimeter compared to the wild type OmpF pore utilizing long side chain hydrophobic resides on one side of the pore and smaller ones on the other, and (c) cork-screw designs (CSD) which introduce a lateral twist as we proceed along the pore axis stemming from alternating stacking long with short side-chain amino acids (FIG. 1).

We were able to redesign the 7×11 Å OmpF WT pore (with molecular retention of solutes with molecular weights of 600 Da or larger) to obtain an array of designs with varying pore size profiles sampling pore sizes across the 3-10 Å range and experimentally tested a subset of these designs in the 3-4 Å range critical for the most challenging separations. The permeabilities of tested designed ranged from 1.0 (±0.23)×10⁻¹² cm³/s for the WT to 4.4 (±0.93)×10⁻¹² cm³/s for the selected CSD design compared to ˜8.97×10⁻¹⁴ cm³/s estimated for AQP1. CSD osmotic water permeabilities were not only higher than AQP1 by two orders of magnitude but were also an order of magnitude higher than the highest reported permeabilities of any channel of the aquaporin family of proteins (2.4 (±0.47)×10⁻¹³ cm³/s determined for AqpZ). Solute rejection capabilities of these channels were evaluated using stopped flow experiments with various solutes. These results demonstrate the potential of computationally tuning the pore diameter of OmpF in response to desired separations.

### Recapitulating Water Wire Geometry of AQP1 in OmpF Mutants

Aquaporins have the ideal internal pore geometry for selective and highly permeable water channels but the pore wall interacts strongly with the permeating water wire, indicating that the possibility of enhancing permeability at similar size ranges without sacrificing selectivity. All aquaporins have a conserved asparagine-proline-alanine motif (known as the NPA motif) near the constriction region, in which the Asn interacts with the water wire by forming hydrogen bonds. These interactions impede the hydraulic permeability through AQP1. In addition to the NPA motif, there are twelve amino acids along the internal pore profile of AQP1 that can form hydrogen bonds with the water wire. Further, the number of hydrogen bonds between the water wire and the inner pore wall of AQPs was directly related to the single channel permeability of the pore. Our aim is to redesign the water channel such that it minimizes interaction with the permeating water wire, thus eliminating hydrogen bonds in the central part of the channel, but retaining the water wire geometry. To discern the unique water wire configurations through AQP1, we examined individual frames of AQP1-water molecular dynamics simulations. Water wires from each simulation frame were isolated and clustered using a k-means approach. Four clusters representing four types of water wire geometries were observed. Even though all four were non-uniform helical water wires, members of the same cluster had their pitch per turn and major and minor axes values that were similar to each other compared to members of another cluster. Each water wire was subsequently positioned inside the OmpF pore and PoreDesigner was used to alter the pore constricting residues to form the equivalent of a molecular “mold” around the water wire (FIG. 1A).

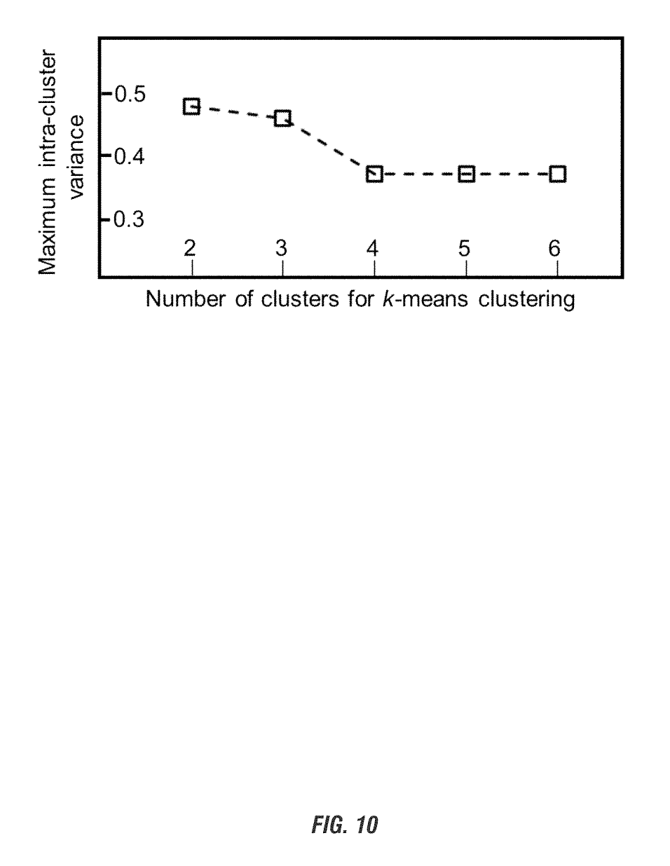

An all-atom 10 ns MD simulation was performed to identify the various geometric poses assumed by the water wire when permeating through AQP1 (FIG. 1) and ˜30,000 frames with ˜1,000,000 water wire trajectories were collected. Thereafter, the principal geometric modes of water transport were determined by clustering the similar water wires using a k-means clustering protocol. All the water wires were observed to assume an elliptic helix shape which can be represented by three parameters—major and minor axes of the ellipse, and pitch per turn. K-means clustering of the ˜1,000,000 water wires yielded four unique water wire geometries (FIG. 10 and Table 2).

### Categorization of the Designs Based on Resultant Internal Pore Geometry

Each of the four unique water wires was placed in the pore of the crystal structure of wild-type OmpF (2omf.pdb) one at a time and used as input into PoreDesigner. Thereafter, the porin and the water wire were aligned such that the constriction center was at the origin and the longitudinal pore axis coincides with the Z-axis. The pore constricting residues were altered to fill up the annular space using hydrophobic, long side-chain amino acids with the objective of designing a narrow yet hydrophobic pore with minimal water-wire interaction. An explicit constraint was imposed to ensure that the distance between any atoms of the pore constriction residue side chain and the water wire oxygen was greater than the sum of their van der Waals radii. This precludes the possibility of arriving at designs with pore constriction residue side chains clashing with the water wire. Hydrogen atoms and atoms from residues away from the pore constriction were excluded from this checklist. PoreDesigner reduces binding with the central water wire by maintaining their respective interaction energies at their maximum by replacing the pore constriction residues of wild-type OmpF with long side-chain, hydrophobic amino acids such as tryptophan (Trp), phenylalanine (Phe), and tyrosine (Tyr). By imposing a minimum percentage (50%) required of long side-chain residues in the redesigned we safeguard against the trivial designs involving all alanines or valines that would be too far to interact with the water wire. We appended a design assessment step at each iteration by accepting only designs whose constriction diameter is less than 4 Å (see Example 2 for calculation).

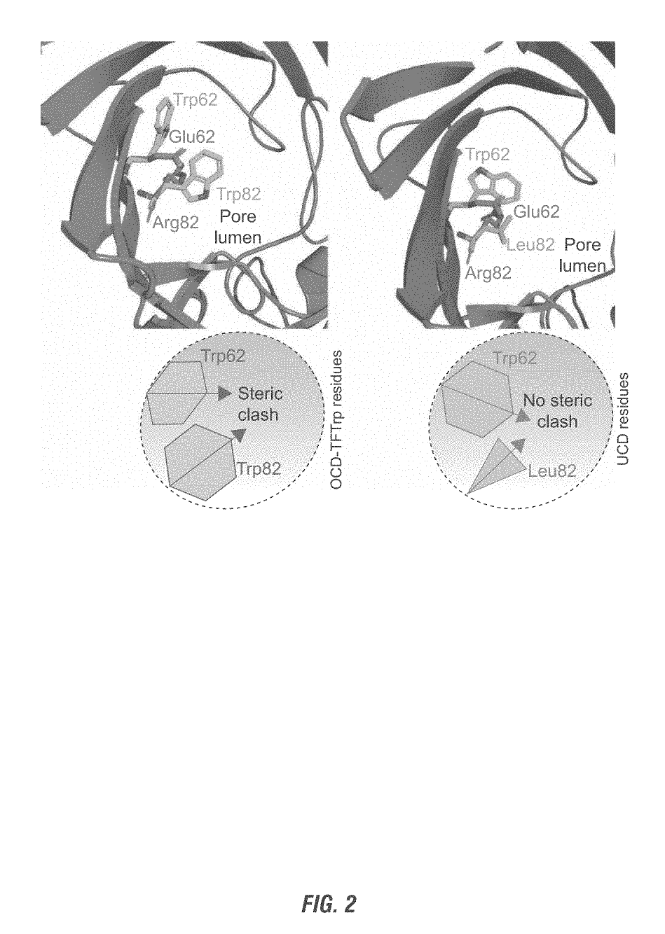

PoreDesigner yielded 40 different OmpF designs with pore sizes less than 4 Å. Analysis of the engineered designs revealed that they conform to three categories based on the resultant internal pore geometry: (1) only Trp/Phe mutations resulting in a narrower but off-center pore lumen (OCD: off-center pore closure design), (2) a smaller co-centric pore with the bulky groups (such as Trp, Phe and Tyr) interspersed with less bulky alanines and valines arranged in a single plane (UCD: uniform pore closure design), and (3) regularly patterned larger with smaller side-groups along the pore profile resulting in an internal pore geometry that involves a twist. We refer to this class of designs as cork-screw designs (CSDs). There were two off-center (OCD) designs for which we allowed the mutation of the 25 pore constriction residues to: (a) only Phe mutations (TFPhe), and (b) only Trp mutations (TFTrp). A biased distribution of bulky groups (Phe/Trps) towards one side of the pore periphery resulted in a smaller pore with its center away from the large residues. The presence of twenty-five Phe/Trps led to steric clashes forcing most of the Phe/Trps side chains to face away from the pore lumen (FIG. 2). Despite the fact that the non-lumen facing Phe/Trps did not contribute to the pore size reduction, they enhanced the inner pore wall hydrophobicity. In the remaining 38 designs, all hydrophobic long side chain amino acids were permitted. These designs sample generally smaller pore sizes by placing long side chain residues interspersed with short amino acids (generally alanines, valines, and leucines). These designs eliminate the possibility of a Phe-Phe/Trp-Trp side-chain steric clash as often seen in the OCD designs (FIG. 1B). We identify these designs as UCD designs. However, there exist a few designs which conform to the third type (CSD) from among these 38 designs. We identified were 31 UCD and seven CSD designs. We chose the smallest predicted pore size design from each type for subsequent hydraulic permeability and solute rejection experiments and molecular dynamics simulations. The predicted pore constriction dimensions after MD simulations for the three selected designs were: 3.54λ3.25 Å, 3.18×3.12 Å, and 3.05λ3.01 Å for OCD, CSD, and UCD protein designs, respectively. These OmpF mutant proteins and the wild type OmpF proteins were produced by expression from synthetic genes cloned into the pET23a(+) expression plasmid vector transformed into E. coli BL21(DE3) Omp8 Rosetta (AlamBompF::Tn5 ΔompAΔompC) mutant strain. The purified proteins were incorporated into liposomes for assessment of single channel water permeability and solute passage as described in subsequent sections.

Before testing the three designs for water transport, the hydrophobicity scores of the three designs were calculated and contrasted with that of wild type OmpF. Table 1 shows the ranges of values for the inner pore wall, outer pore wall, and overall free energies of transfer from water to ethanol (which serve as a surrogate to hydrophobicity scores) of the three selected OmpF mutants in contrast with the wild type. The respective hydrophobicities were computed by adding up free energy of transfer (from water to ethanol) of each of the amino acid side chains that constitute the inner and outer pore wall. A higher negative value represents a higher hydrophobicity. This scale reports the free energies of transfer of different amino acid side chains from water phase to ethanol. As a result, the hydrophobic amino acids have a lower free energy of transfer than charged amino acids. The relative order of inner pore wall hydrophobicities was seen to be CSD>OCD>UCD>wild type Ompf. We hypothesized that the experimentally measured single channel permeabilities will follow the same trend as increasing hydrophobicity based on our design principle of eliminating water-pore wall interactions to enhance permeability.

TABLE 1

| | Inner pore wall | Outer pore wall | Overall |
|---|---|---|---|
| | hydrophobicity | hydrophobicity | hydrophobicity |
| | score | score | score |
| | ΔG_{transfer}^{water→ethanol} | ΔG_{transfer}^{water→ethanol} | ΔG_{transfer}^{water→ethanol} |
| Designs | (kcal mol⁻¹) | (kcal mol⁻¹) | (kcal mol⁻¹) |
| CSD | −97.7 | −76.5 | −174.2 |
| OCD | −69.2 | −81.2 | −150.4 |
| UCD | −65.6 | −71.3 | −136.9 |
| wild | −61.3 | −68.5 | −129.8 |
| type | | | |

The hydrophobicity trends reveal that the OCD-TFTrp mutant has the highest estimated outer pore wall hydrophobicity. This is possibly due to the steric clashes between contiguous tryptophans (FIG. 2) where the majority of the 25 pore-constricting tryptophans are forced away from the lumen.

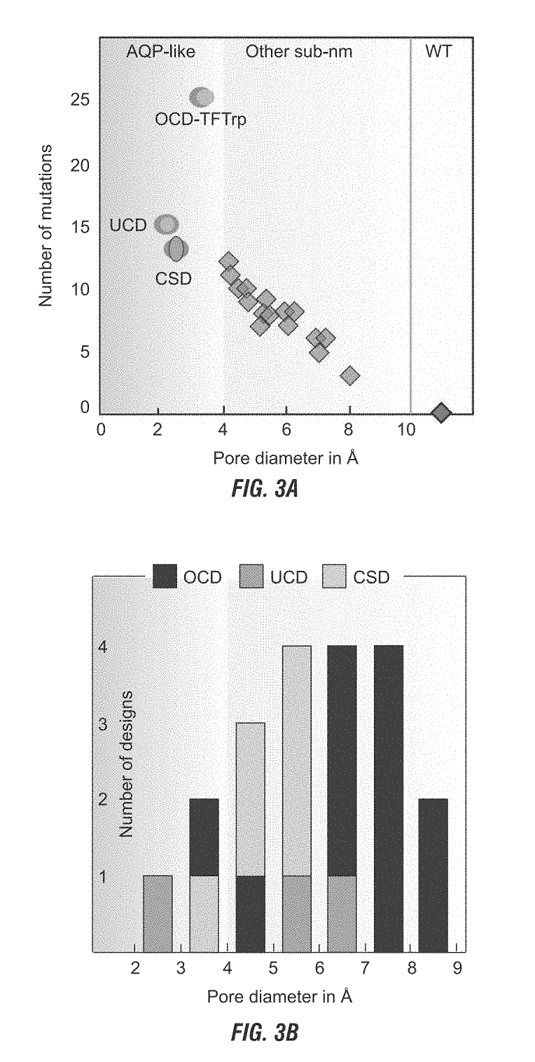

In addition, we also used PoreDesigner to predict designs that span the remaining 4-10 Å range. The overall goal is to precisely match any desired pore size needed for separations spanning the sub-nm (3-10 Å) range. PoreDesigner was accordingly modified to only accept pore designs with pore constriction diameter between a pre-specified range D^{min} and D^{max}. For example, setting D^{min} and D^{max} values to 5 Å and 6 Å respectively, yields OmpF designs with pores predicted to be within this range. We identified 17 new designs (FIG. 3) with at least two designs within a pore size bin of range 1 Å starting from 4 Å to 9 Å. We used the aforementioned structural classification scheme and developed ten OCD designs, three UCD and four CSD designs. Generally, the smaller the desired pore size, the higher was the number of required mutations. OCD type designs were seen to be most prevalent spanning almost the entire sub-nm range (FIG. 3B). Whereas, CSD and UCD type designs were limited to the mid region of the sub-nm spectrum.

### Experimental Validation of Pore Designs

The wild type OmpF has a pore size around 7×11 Å and a molecular weight cutoff of around 600 Da. We redesigned OmpF to target a range of sub-nm pore sizes as discussed above. We selected one mutant from each class of designs targeting sub 4 Å pore size measured their single channel permeability and solute passage rates experimentally. The three OmpF mutants that we chose were OCD with in silico estimated pore sizes of 3.25 Å (minor axis of elliptical pore cross-section), CSD with pore size of 3.12 Å, and UCD with estimated pore size of 3.01 Å.

OmpF can be reconstituted into lipid vesicles and allows passive diffusion of small molecules across the membrane. We characterized the influx of different molecular weight solutes through wild type OmpF and its mutants under hypertonic conditions using stopped flow light scattering measurements, and compared their solute transport trends. All OmpF mutants were reconstituted into L-α-phosphatidylcholine(PC)/L-α-phosphatidylserine (PS) vesicles using a detergent destabilization method at a lipid to protein mass ratio 400 (LPR400). The liposomes were rapidly mixed with hypertonic solutions in the mixing cell of a stopped flow setup, NaCl, glycine, glucose, sucrose, and polyethylene glycol 600 (PEG600) as osmolytes.

### Solute Rejection of OmpF Mutants

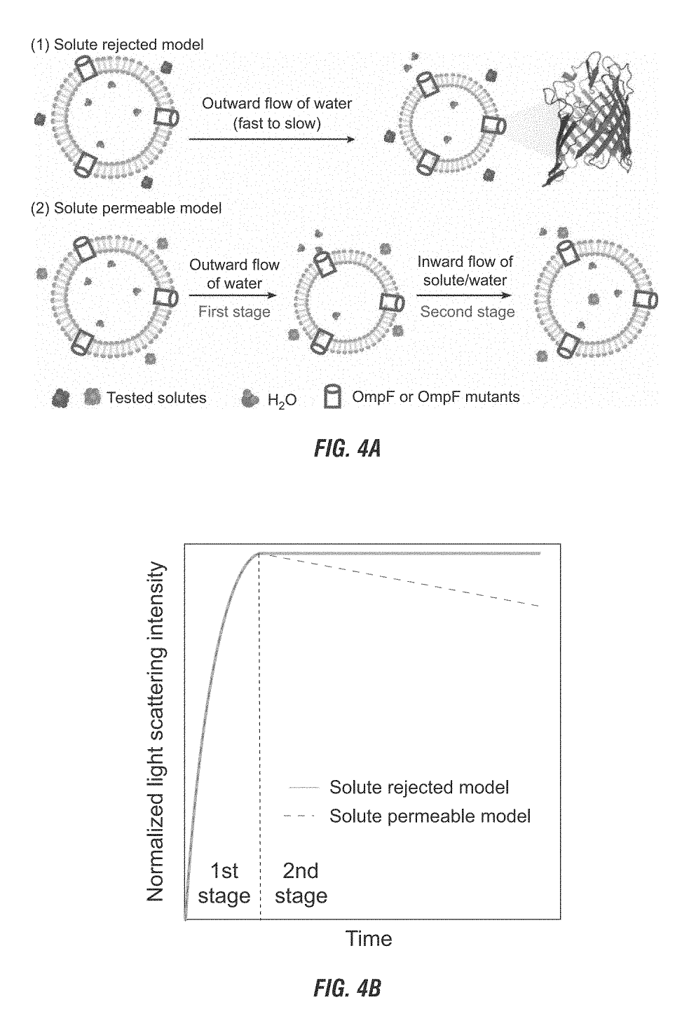

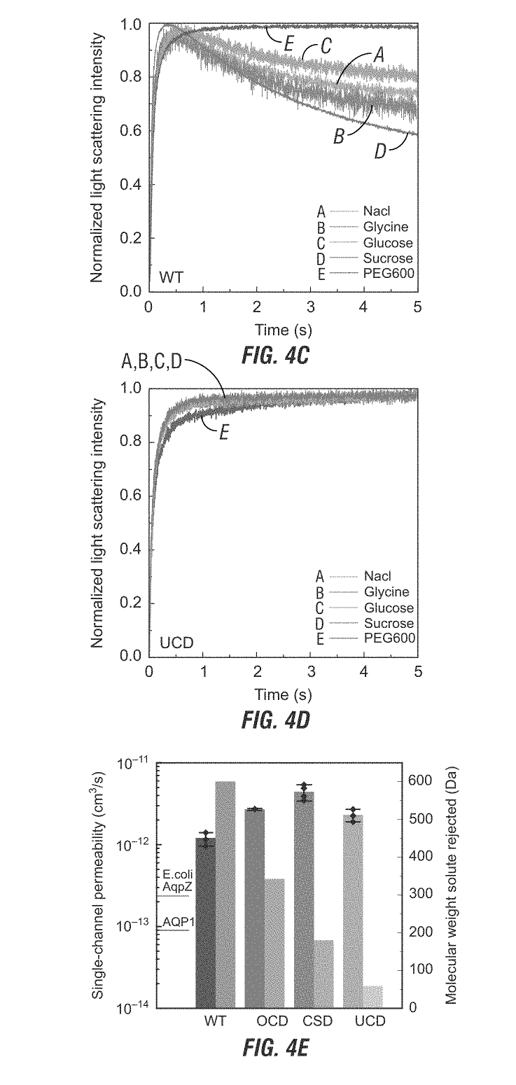

In the method employed, proteoliposomes are subjected to a hyperosmotic shock, and time dependent light scattering data collected upon mixing of the osmolyte and proteoliposmes. The resulting light scattering profile can be used to determine solute exclusion as well as water permeability of incorporated proteins. As shown in FIG. 4A, for both the cases where solute is completely excluded by the channel (solute exclusion model) and when there is some solute leakage through the channel (solute permeable model), during the first stage of the mixing process water flows outward from the vesicles leading to vesicle shrinkage, due to the high osmolarity outside the vesicles. This shrinkage leads to an increase of light scattering intensity measured at 90 degrees to the incident light due to constructive interference of scattered light. This is because vesicles with a size comparable to the wavelength of light stop acting like point particles and show an increasing trend in scattering intensity with decreasing volume at the scattering angle used for measurements (90°). During the second stage of the mixing process (FIG. 4B), the light scattering intensity trend changes based on whether the solute can or cannot not diffuse through the porin. When the solute molecular size is larger than the porin pore size (FIG. 4B), there would be inflow of water and solutes into vesicles, and the light scattering intensity levels off as measurement time increases. However, if the solute size is smaller than the porin pore size (FIG. 4B), solutes diffuse through porins and led to a corresponding influx of water into vesicles, which is observed as a decrease in light scatting intensity. Based on the observation of light scattering intensity change, we can estimate the solute rejection trends of porins.

We estimated the approximate molecular weight limit at which solute rejection for WT OmpF and the three OmpF mutants occurred. For WT OmpF, the light scattering intensity decreased at the second stage when WT OmpF reconstituted liposomes were exposed to NaCl (or glycine, glucose, and sucrose) containing hypertonic solutions, which indicates that WT OmpF is permeable to these solutes. The light scattering intensity leveled off when exposing the proteoliposomes to PEG600 containing hypertonic solutions (FIG. 4C). This observation demonstrated that WT OmpF can reject PEG600 (600 Da) or larger molecules, which is consistent with previous reports.

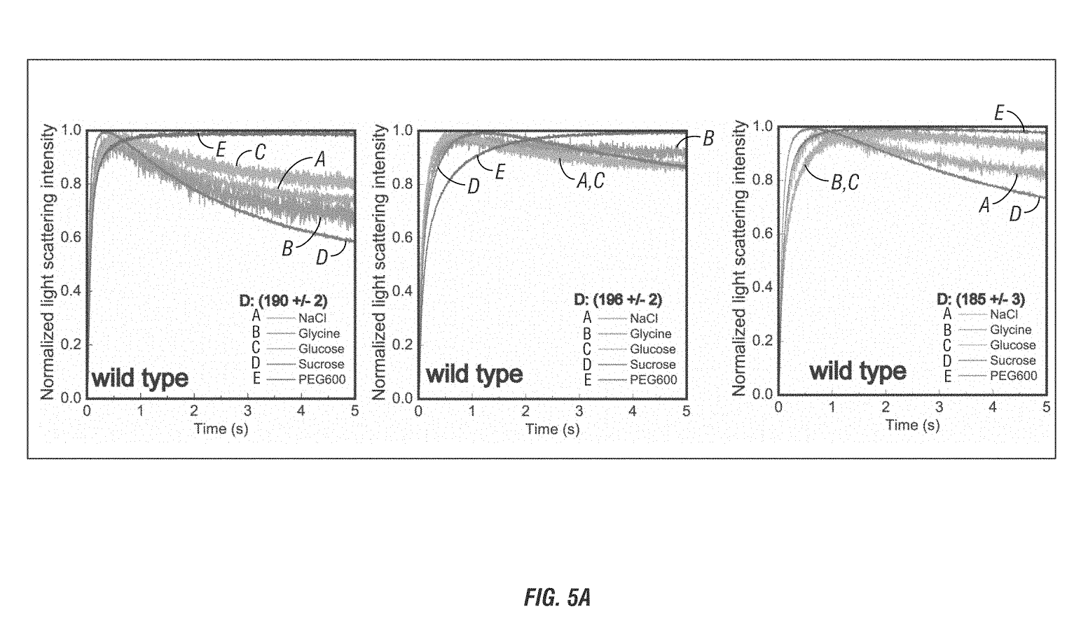

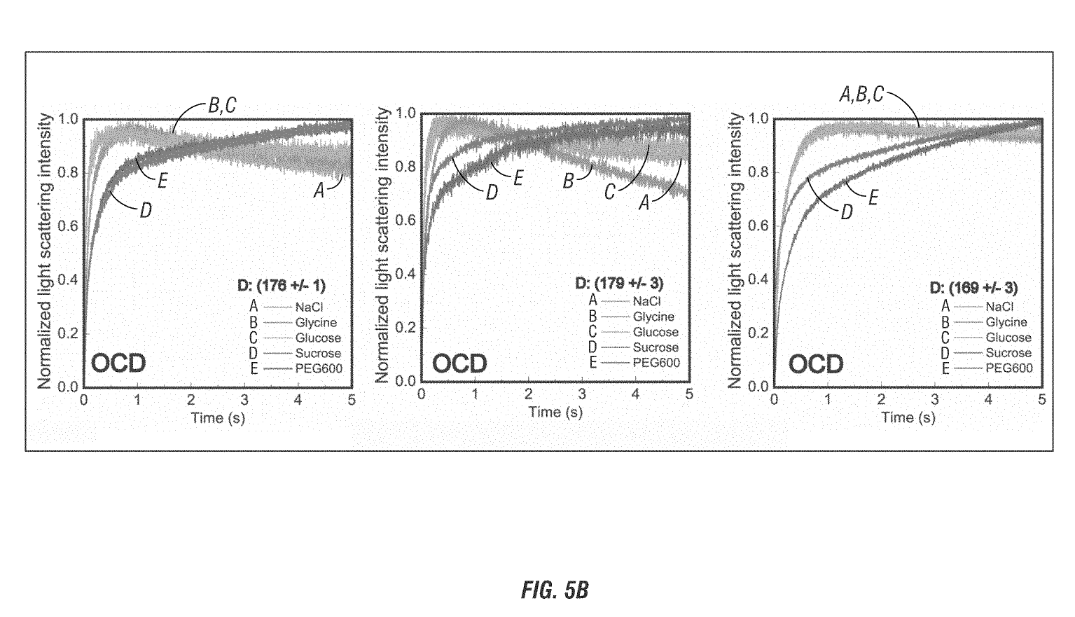

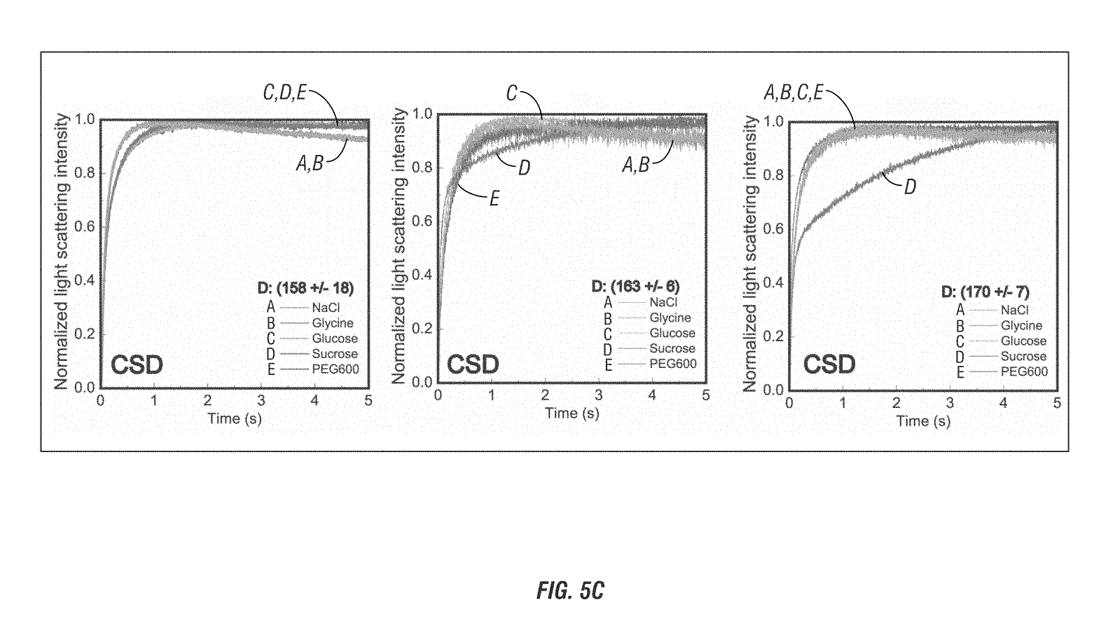

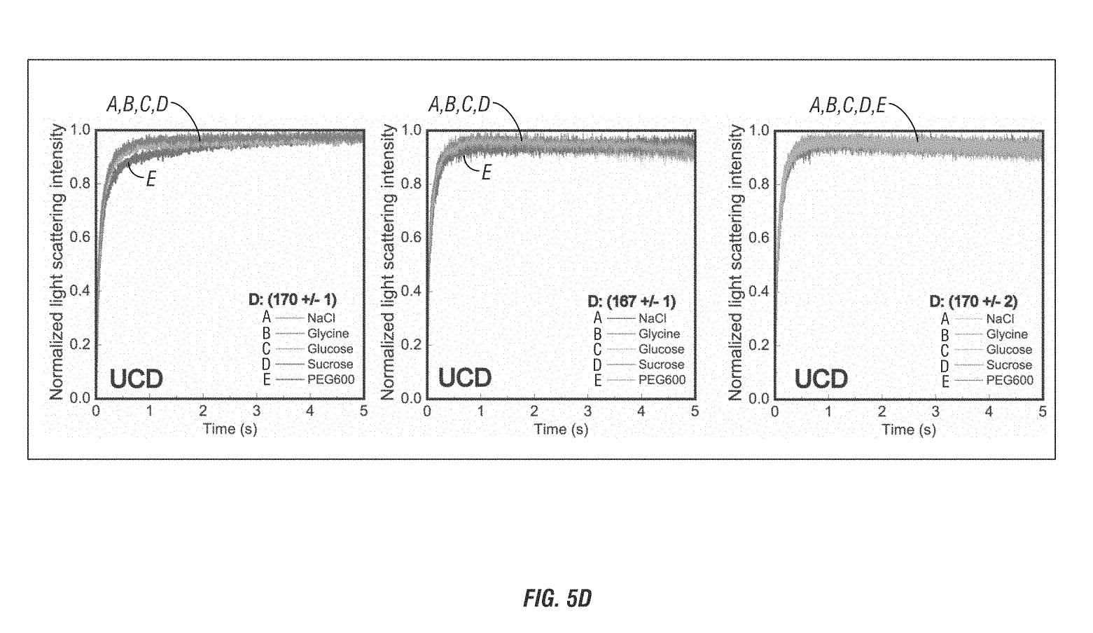

For UCD, the light scattering intensity leveled off when exposing the liposomes to all the solutes used including NaCl (58.5 Da), leading us to conclude that this mutant can substantially reject molecules larger than 58.5 Da (FIG. 4D). We also estimated the approximate molecular weight exclusion limit for OCD and CSD (FIG. 5). Based on the solute rejection experiments above, we estimated the molecular exclusion limit of the three mutants to have the following sequence: Wild type (˜600 Da)>OCD (˜342 Da)>CSD (˜180 Da)>UCD (˜58 Da) (FIG. 4E) which follows the same trend as the designed pore sizes. Thus, small molecule separation membranes can be developed by a selection of different pore size OmpF mutants for biomimetic membranes. The mutant with the smallest pore size, UCD, has ionic solute rejection properties similar to aquaporins (while not excluding protons), and can be selected as a candidate protein for developing membrane protein based biomimetic desalination membranes.

### Single Channel Permeability of OmpF Mutants

Recent literature has focused on emphasizing the importance of membrane design efforts that lead to high selectivity while maintaining or increasing current membrane permeabilites. Solute rejection experiment results showed that high molecular selectivity can be achieved by designing OmpF mutants with different pore sizes through the PoreDesigner workflow. In addition to estimating selectivity, we also evaluated OmpF mutant permeabilities, which were specifically characterized by determining single channel permeability of each mutant.

The permeability values of vesicle membranes containing various mutant OmpF proteins were calculated from the light scattering intensity curves obtained from stopped flow light scattering experiments conducted with completely the excluded solute PEG 600 as an osmotic agent for all mutants. By fitting the normalized light scattering intensity curve to a double exponential curve similar to that was used in previous work we obtained a rate constant k (the larger constant from the double exponential fit). This rate constant was then used to calculate the osmotic water permeability (P_{f}) using the following equation:

$${P}_{f} = \frac{k}{( \frac{S}{{V}_{o}} ) \times Δ π \times {V}_{w}}$$

where S is the initial surface area of OmpF reconstituted vesicles, Vₒ is the initial volume of OmpF reconstituted vesicles, Δπ is the osmotic gradient across the lipid bilayers, and V_{w} is the molar volume of water.

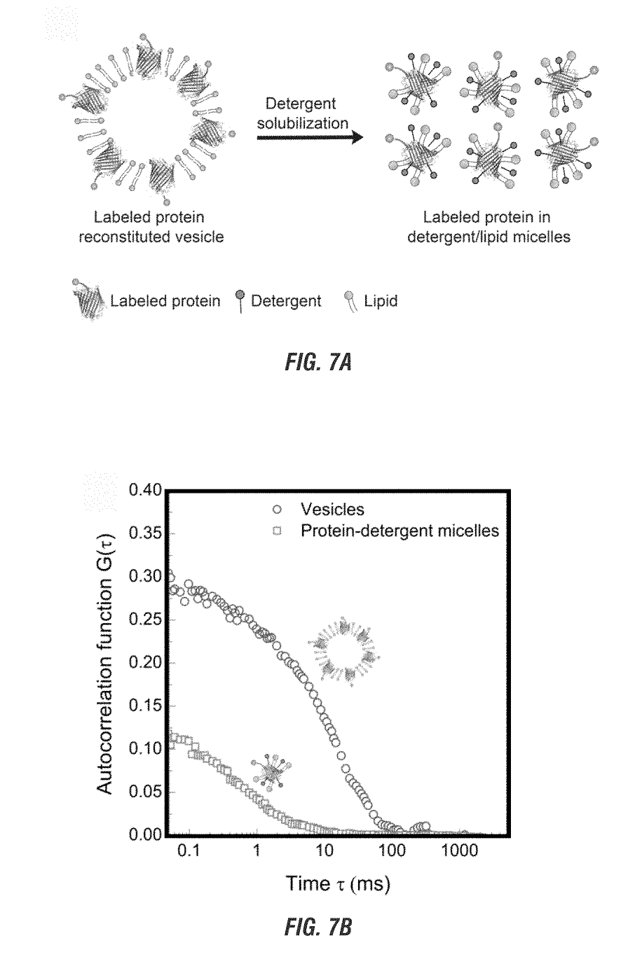

FIG. 4 shows light scattering curves obtained from vesicle membranes with reconstituted WT and mutant OmpF proteins at a lipid to protein mass ratio of 400 (LPR400), with net permeabilities between 2049 and 3411 μm/s. Because net permeability can depend both on the number of proteins reconstituted and the single channel permeability, for more accurate comparison between mutants, we calculated the single channel permeability of wild type OmpF and its mutants. Single channel permeability was calculated by combining the net permeability from stopped flow light scattering measurement with the number of proteins inserted per vesicle, determined from fluorescence correlation spectroscopy (FCS) experiments. This approach has been used to calculate the single channel permeability of Aquaporin Z and peptide-appended pillar[5]arene (PAP) channels successfully. OmpF proteins were first labeled using a pyrylium dye, which is shown to only have detectable fluorescence signal after conjugation with proteins. We reconstituted these labeled OmpF proteins into vesicles and performed FCS to first determine the number of vesicles (Nᵥₑₛ) by fitting the auto correlation function obtained from FCS measurements of these fluorescent vesicles. We then added the membrane protein compatible detergent, octyl glucoside (OG) to the same vesicle solutions (final OG concentration is 2.5%) to break down the vesicles into protein-detergent micelles. Thus, we can calculate the number of proteins (Nₚᵣₒ) using the same method by conducting FCS measurements on these solubilized proteins assuming one protein trimer per micelle similar to what has been reported for aquaporins. By taking the ratio of the number of proteins to the number of vesicles, we can obtain the average number of proteins per vesicle (Nₚᵣₒ/Nᵥₑₛ) (FIG. 7). Combining vesicle permeability and average number of OmpF proteins per vesicle, we calculated average single channel permeability of OmpF and its mutants (FIG. 4E). CSD had the highest single channel permeability followed by OCD and UCD, which have similar single channel permeabilities and all the three mutants have single channel permeabilities higher than WT OmpF. This serves as the experimental corroboration of the predicted water permeation rates from the inner pore wall hydrophobicities. Compared to aquaporins, single channel permeabilities of wild type OmpF and its mutants are at least an order of magnitude higher, the highest single channel water permeability of OmpF mutants is ˜18 times faster than that of the E. coli AqpZ, which was measured using the same platform and ˜49 times faster than that of AQP1.

### Molecular Dynamics Simulation of the Pore Designs

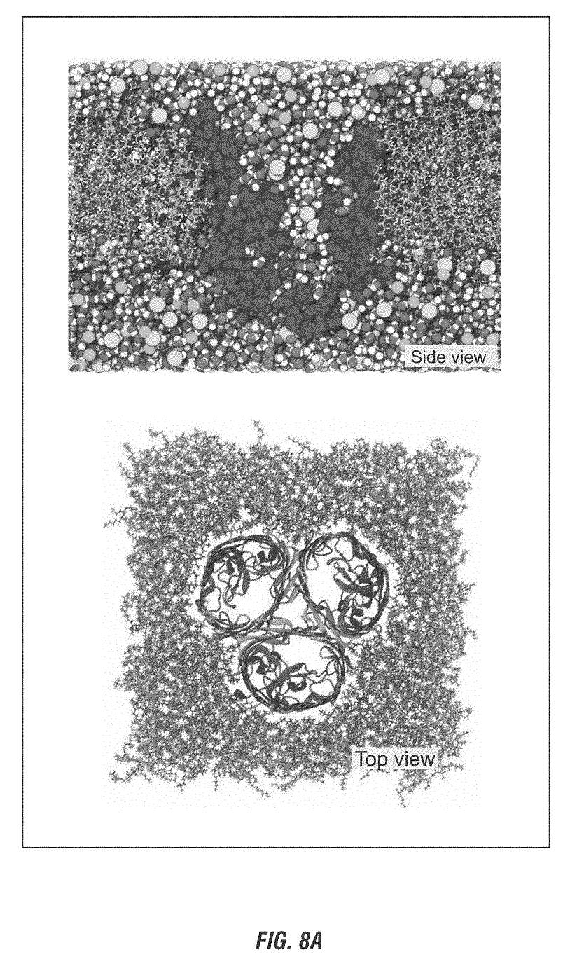

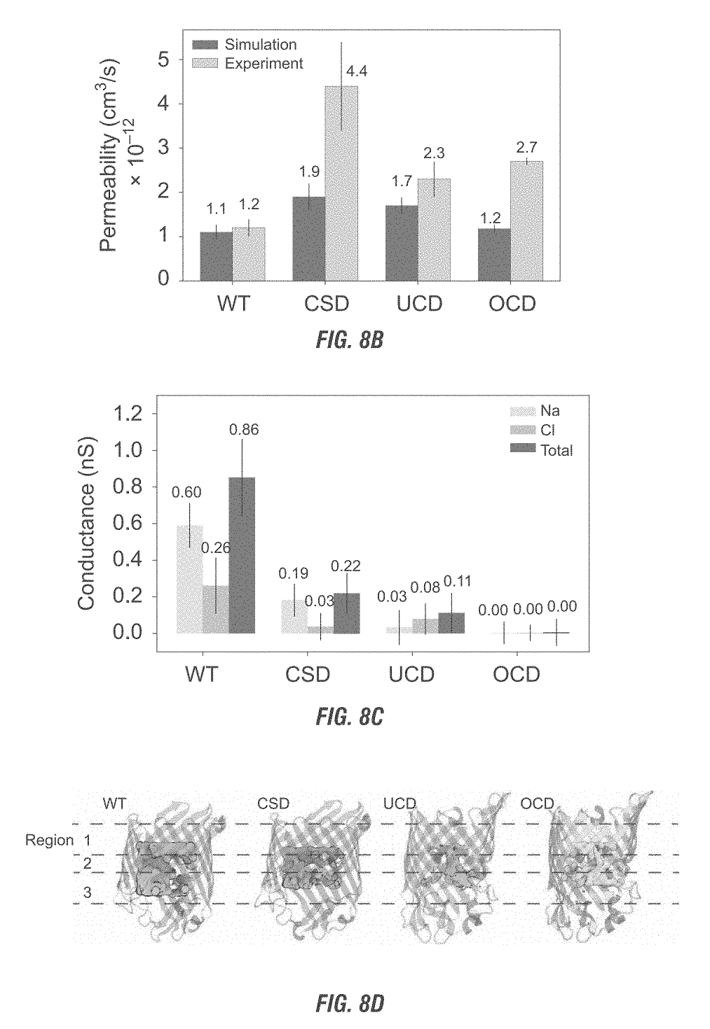

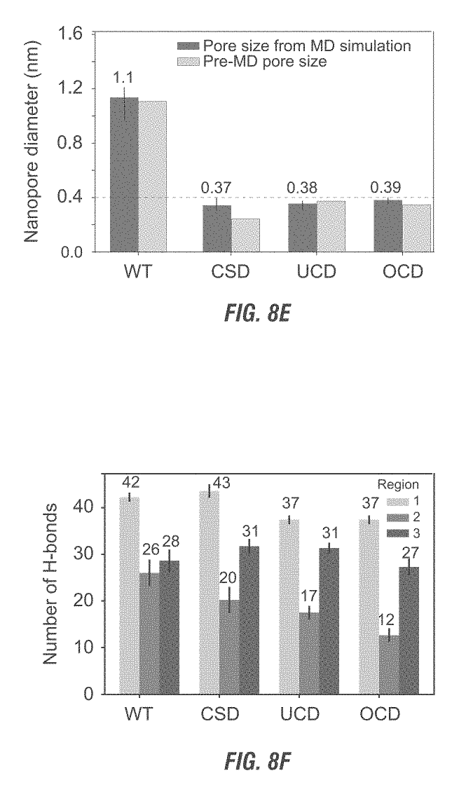

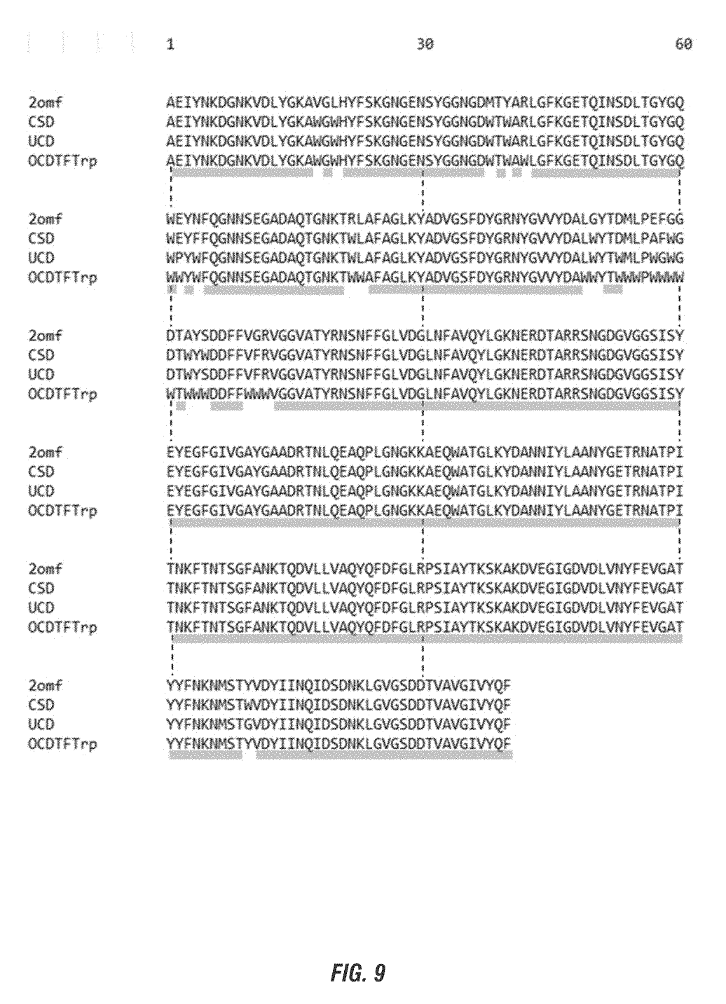

Using the all-atom MD method, we independently assessed osmotic permeability of the wild type OmpF and the three experimentally verified designs, starting from the molecular configurations suggested by PoreDesigner. The monomeric proteins were patched to form trimers using a VMD plugin, set in a lipid-bilayer and solvated in 1 M NaCl solution. (FIG. 8A). Osmotic permeabilities were evaluated (from the rate of water displacements) and averaged through each monomer of the trimeric molecule and the variabilities during the last 30 ns of the 35 ns simulation were reported (FIG. 8B). The MD-computed osmotic permeabilities of OmpF and the three designs are seen to corroborate the same single channel permeability trend as seen in stopped-flow light scattering experiments. Highest permeability of CSD reaffirms the applicability of using inner pore wall hydrophobicity scores as a surrogate to predict relative channel permeabilities.

The ionic conductances of the WT and mutant pores were evaluated by simulating the systems under a transmembrane voltage of 500 mV (FIG. 8C). The ionic conductance was determined by averaging instantaneous displacements of ions over the last 25 ns of the 35 ns MD trajectories. All the three mutants exhibited negligibly low conductances which were about an order of magnitude lower than that of wild type OmpF with the CSD mutant being the most conductive of the three as expected from solute rejection experiments. While solute rejection experiments identified UCD to be more restrictive towards salt compared to OCD, MD simulations, as conducted, do not seem to be conclusive regarding the difference between the two mutants (FIG. 8C).

Further analysis of the MD trajectories identified the inner volume of the WT and mutant pores accessible to water (FIG. 8D). The volumes were determined by averaging water density over the last 10 ns of the MD simulations carried out under a 500 mV bias. The volume density maps reveal the UCD mutant to have the narrowest pore constriction, which correlates with the best solute rejection performance of that mutant. FIG. 8E shows the major pore constriction diameters as measured using PoreDesigner (before MD simulations were performed) along with that observed during the 5 ns of MD. The closely packed side chains of OCD and UCD allowed marginal movement of pore constriction amino acid side chains, thereby showing less variability in the pore size during the course of MD. However, the bulky groups of CSD are stacked in different planes thus allowing some movement of the pore constriction side chains leading to higher variability in pore sizes during water permeation. The same PoreAnalyzer that was used in PoreDesigner was used for assessing the pores from the MD trajectories.

To assess the effect of relative hydrophobicity of the mutant pores, we determined the average number of hydrogen bonds between the OmpF monomer and water located in each of the three regions of the pores (FIG. 8D). A hydrogen bond was reported if the water molecule was within 0.3 nm of a protein atom capable of forming a hydrogen bond. It was also ensured that the protein atom-water hydrogen-water oxygen angle was 20 degrees or less. The number of calculated bonds was averaged over entire 35 ns MD trajectories with error bars showing standard deviations (FIG. 8F). The constriction region (FIG. 8F) shows progressively decreasing number of hydrogen bonds as the number of mutations (in the constriction region) as the pore is occluded by more hydrophobic residues. This attests to the effectiveness of PoreDesigner's design objective of replacing pore constriction residues with hydrophobic ones to limit pore wall-water wire interactions in order to arrive at designs with high single channel permeabilities while tuning size selectivity.

### DISCUSSION

Ultrapermeable membranes (dense solution, diffusion-based or channel-based) have emerged as a promising alternative to energy-intensive separations including desalination and water purification. AQPs are ideal candidates for channel-based membranes owing to their high permeabilities and selectivities but the range of solutes that can be separated are limited.

Here, we have successfully put forth a computational (i.e., PoreDesigner) and experimental workflow that relies on the mechanically, chemically, and mutationally stable beta-barrel scaffold of OmpF as a candidate for synthesizing channel-based membranes for speedy aqueous-phase separations of specific solutes, thus expanding the selectivity range of current AQP-based biomimetic membranes.

The use of OmpF provides two distinct advantages over the use of AQPs for the envisioned applications in aqueous separations. First, AQPs are arguably overdesigned for water desalination as they remove protons along with other monovalent and divalent ions while OmpF can be designed to pass protons while removing other ions. The requirement to reject protons imposes the need to have hydrogen bonding between translocating water molecules and the pore wall. Recent studies have indicated that an ideal water-conducting pore could transport water more efficiently if all hydrogen bonding between waters and the central section of the pore are eliminated. Second, the higher permeability of OmpF over AQPs can be advantageous for water purification and desalination in specific instances where space is at a premium or where energy savings can be substantial. Ultrapermeable membranes, such as those based on OmpF, with high salt rejection suited for RO can considerably minimize energy requirements (˜45%) or plant infrastructure (pressure vessels, up to 65%) in streams with modest salinity; for instance brackish water desalination and recycling. Even though the energy advantage is marginal for high salinity seawater applications (15% less energy), there is a considerable plant size reduction (i.e., 44%). Sub-nm pore size membranes for nanofiltration (NF) and ultrafiltration (UF) have diverse applications in water treatment, food production and processing, and energy applications which will also benefit from energy and capital cost reduction.

We demonstrated that PoreDesigner can precisely design sub-nm pore sizes within the stable beta barrel of a bacterial channel porin without jeopardizing the channel stability. PoreDesigner provides another powerful demonstration of de novo protein design on a class of proteins that so far have not been the subject of systematic protein design as have enzymes, antibodies, binding proteins or protein interfaces. We experimentally validated our designs and show excellent solute selectivity for a range of solutes while maintaining high permeabilities.

We subsequently expanded upon the range of designs obtained by PoreDesigner with pore sizes spanning the entire sub-nm (3-10 Å) spectrum in 1 Å bin sizes. PoreDesigner (FIG. 3) identified designs that spanned the entire pore size spectrum. OCD designs were seen to span the entire sub-nm spectrum whereas CSD and UCD designs were limited to the mid of the sub-nm spectrum. Successful computation-driven designs for the 4 Å range suggest that the proposed framework lays the foundation of a new paradigm for membrane-based sub-nm aqueous separations with applications ranging from desalination, vesicle-mediated drug delivery to separating solutes of biochemical importance with marginal difference in sizes. Moving beyond OmpF, PoreDesigner can be applied to tune pore size and geometry for any other porin system, particularly those with a known structure.

### EXPERIMENTAL METHODS

OmpF and the three mutant design proteins were produced by homologous expression of synthetic genes using pET23a(+) in an E. coli BL21(DE3) Omp8 Rosetta mutant (Genotype: F− ompT hsdSB(rB−mB−) gal dcm (DE3) pRARE (CamR) ompR ΔlamB ompF::Tn5 (KanR)) strain according to the pET cloning and expression system (Novagen). Thereafter the porins were purified in their native trimeric state and stabilized in a detergent solution before reconstitution, and subsequently passed through an equilibrated anion exchange chromatography column (HiScreen DEAE HF), and a Superose 12 size exclusion column. Bradford assays were used to determine the protein concentration. Dry lipid films and a rehydration buffer were used to reconstitute mutant and WT OmpF which were extruded using a 200 nm track-etched membrane. Stopped-flow light scattering goniometer setup was used to assess hydraulic permeabilities. Finally, high concentrations of various solutes of different hydrodynamic radii were used in the mixing cell of the apparatus to determine solute rejection performance of the mutants in contrast to the WT OmpF.

### OmpF Expression and Cell Culture

Wild-type OmpF and OmpF mutant proteins were produced using the pET cloning and expression system (Novagen). Gene sequences for the WT and mutant OmpF protein designs were codon optimized, synthesized and cloned by GenScript USA (Piscataway, N.J.). The synthesized genes were cloned into the NdeI-XhoI sites of the pET23a(+) expression vector and plasmids were maintained in E. coli TOP10. Purified plasmids were transformed into E. coli BL21(DE3) Omp8 Rosetta (AlamBompF::Tn5 ompA ompC) mutant strain. Four transformant strains were isolated: OmpF-WT, OmpF-OCD, OmpF-CSD, and OmpF-UCD. Each strain was grown in Luria-Bertani (LB) with Cloramphenicol (20 mg/L) and Ampicillin (100 mg/L) at 37° C. (in batch culture). Once OD600 reached 0.5˜0.7, Isopropyl beta-D-1-thiogalactopyranoside (IPTG) was introduced to the cell culture media at final IPTG concentration of 0.4 mM, then the incubation temperature was decreased to 16° C. for gene expression and protein production. After 12-16 h of cell growth, E. coli cells can be harvested and stored at −80° C.

### OmpF Purification

OmpF mutants were purified and 10 g frozen cells were first dissolved in 100 mL lysis buffer (20 mM Tris, 0.1 mg/mL DNase I, pH8.0) and lysed with an ultrasonic homogenizer. The mixture was spun down at 4,000 g for 20 mins to separate unbroken cells. The lysedcells (in lysis buffer) were mixed with SDS at a final SDS concentration of 0.5% (wt/v) for 20 mins at 4 degree C. and centrifuged at 200,000 g for 60 mins to harvest cell membrane pellets. Cell membrane pellets were suspended in 0.125% Octyl-POE, 20 mM Na₃PO₄, pH7.4 buffer (50 mL buffer/10 g original cells), and then cell membrane pellet mixture was incubated at 37 degree C. for 60 mins. Then the membrane pellet mixture was centrifuged at 200,000 g for 60 mins in a Thermo Sorvall WX ultracentrifuge. The pellet was resuspended in 3% Octyl-POE, 20 mM Na₃PO₄, pH7.4 buffer (25 mL buffer/10 g original cells), and the mixture was incubated at 4° C. overnight. On the second day of the purification, the mixture was incubated at 37° C. for one hour before ultracentrifugation (200,000 g, 30 mins) to spin down unsolubilized cell membranes. The supernatant was collected after ultracentrifugation for chromatographic separation.

In the next step, an anion exchange chromatography column was used (HiScreen DEAE FF). This column was first equilibrated with 5 mM Na₃PO₄, 1% Octly-POE, 3 mM NaN₃, pH7.6 buffer. Then the supernatant previously collected was loaded into the column and eluted with 5 mM Na₃PO₄, 1% Octly-POE, 3 mM NaN₃, 30 mM EDTA, 100 mM NaCl, pH7.6 buffer. The elution peak fractions were combined and loaded onto Superose 12 size exclusion column (equilibrated with 10 mM Tris, 0.1M NaCl, 1.2% Octyl glucoside) to as a final purification step. The size exclusion peak fractions were collected for further use. Protein concentration was measured by Bradford assay.

OmpF Reconstitution into Vesicles

For OmpF reconstitution into liposomes, we used a previously reported “detergent destabilization” method. Briefly, vesicles were created by rehydrating dry lipid films in rehydration buffer (20 mM HEPES, 100 mM NaCl, 0.02% (wt/v) NaN₃, pH7.4) and then extruded through 200 nm track-etched membranes. To these monodisperse vesicles predetermined amounts of 10% (wt/v) Decyl Maltoside and rehydration buffer were introduced to “loosen” the vesicles to allow for efficient membrane reconstitution. OmpF protein was added to the detergent/vesicles mixture for reconstitution, and detergent in the mixture was removed by a adding predetermined amount of Bio-Beads' SM-2 resin. After detergent removal, the liposomes can be used for stopped flow experiments.

### Stopped Flow Experiments for OmpF (and Mutants) Solute Rejection Estimation

OmpF (or OmpF mutants) reconstituted vesicle solute rejection was estimated using experiments on a Stopped flow light scattering (SFLS) instrument, based on a well-established protocol for previous ion permeability studies and aquaporin permeability studies. Vesicles were rapidly mixed with a high solute concentration osmotic agent (based on different tested solutes) in the mixing cell of this instrument. The high solute concentration osmotic agent included: 20 mM HEPES, 110 mM NaCl, 0.02% (wt/v) NaN₃, pH7.4 (for NaCl rejection measurement); 20 mM HEPES, 100 mM NaCl, 20 mM Glycine, 0.02% (wt/v) NaN₃, pH7.4 (for glycine rejection measurement); 20 mM HEPES, 100 mM NaCl, 20 mM glucose, 0.02% (wt/v) NaN3, pH7.4 (for glucose rejection measurement); 20 mM HEPES, 100 mM NaCl, 20 mM sucrose, 0.02% (wt/v) NaN3, pH7.4 (for sucrose rejection measurement); 20 mM HEPES, 100 mM NaCl, 15 mM PEG600, 0.02% (wt/v) NaN3, pH7.4 (for PEG600 rejection measurement). Here, we used 15 mM PEG600 in PEG600 rejection measurement experiments to keep the same buffer osmolarity as other high solute concentration osmotic agents (measured in a freezing point osmometer).

Sequences

>2omf (SEQ ID NO: 1)
AEIYNKDGNKVDLYGKAVGLHYFSKGNGENSYGGNGDMTYARLGFKGETQ
INSDLTGYGQWEYNFQGNNSEGADAQTGNKTRLAFAGLKYADVGSFDYGR
NYGVVYDALGYTDMLPEFGGDTAYSDDFFVGRVGGVATYRNSNFFGLVDG
LNFAVQYLGKNERDTARRSNGDGVGGSISYEYEGFGIVGAYGAADRTNLQ
EAQPLGNGKKAEQWATGLKYDANNIYLAANYGETRNATPITNKFTNTSGF
ANKTQDVLLVAQYQFDFGLRPSIAYTKSKAKDVEGIGDVDLVNYFEVGAT
YYFNKNMSTYVDYIINQIDSDNKLGVGSDDTVAVGIVYQF
>CSD (SEQ ID NO: 2)
AEIYNKDGNKVDLYGKAWGWHYFSKGNGENSYGGNGDWTWARLGFKGETQ
INSDLTGYGQWEYFFQGNNSEGADAQTGNKTWLAFAGLKYADVGSFDYGR
NYGVVYDALWYTDMLPAFWGDTWYWDDFFVFRVGGVATYRNSNFFGLVDG
LNFAVQYLGKNERDTARRSNGDGVGGSISYEYEGFGIVGAYGAADRTNLQ
EAQPLGNGKKAEQWATGLKYDANNIYLAANYGETRNATPITNKFTNTSGF
ANKTQDVLLVAQYQFDFGLRPSIAYTKSKAKDVEGIGDVDLVNYFEVGAT
YYFNKNMSTWVDYIINQIDSDNKLGVGSDDTVAVGIVYQF
>UCD (SEQ ID NO: 3)
AEIYNKDGNKVDLYGKAWGWHYFSKGNGENSYGGNGDWTWARLGFKGETQ
INSDLTGYGQWPYWFQGNNSEGADAQTGNKTWLAFAGLKYADVGSFDYGR
NYGVVYDALWYTWMLPWGWGDTWYSDDFFVFRVGGVATYRNSNFFGLVDG
LNFAVQYLGKNERDTARRSNGDGVGGSISYEYEGFGIVGAYGAADRTNLQ
EAQPLGNGKKAEQWATGLKYDANNIYLAANYGETRNATPITNKFTNTSGF
ANKTQDVLLVAQYQFDFGLRPSIAYTKSKAKDVEGIGDVDLVNYFEVGAT
YYFNKNMSTGVDYIINQIDSDNKLGVGSDDTVAVGIVYQF
>OCD-TFTrp (SEQ ID NO: 4)
AEIYNKDGNKVDLYGKAWGWHYFSKGNGENSYGGNGDWTWAWLGFKGETQ
INSDLTGYGQWWYWFQGNNSEGADAQTGNKTWWAFAGLKYADVGSFDYGR
NYGVVYDAWWYTWWWPWWWWWTWWWDDFFWWWVGGVATYRNSNFFGLVDG
LNFAVQYLGKNERDTARRSNGDGVGGSISYEYEGFGIVGAYGAADRTNLQ
EAQPLGNGKKAEQWATGLKYDANNIYLAANYGETRNATPITNKFTNTSGF
ANKTQDVLLVAQYQFDFGLRPSIAYTKSKAKDVEGIGDVDLVNYFEVGAT
YYFNKNMSTYVDYIINQIDSDNKLGVGSDDTVAVGIVYQF

### Example 2: Description of PoreDesigner

PoreDesigner uses results from molecular dynamics simulations (all atom 10 ns with 2 fs timestep) of pressure driven water transport through tetrameric AQP1-membrane assembly. The tetrameric AQP1 contributes four water wires per frame. Thus, 30,000 frames were used to glean ˜1,20,000 water wires. However, nearly 20,000 wires could not be used for the analysis because they were incomplete (i.e. a gap of more than 4 Å between any two contiguous water molecules).

### Isolation of Water Wires

We isolated water-wire trajectories from all-atom 10 ns MD simulations of water permeation through AQP1 using TCL scripts written and executed using Visual Molecular Dynamics (VMD) molecular graphics software. We create an ensemble of water wires from ˜30,000 trajectories thereby sampling a large number of possible geometries of the single file of water through AQP1. We observed that all the water wires assume a spiral path which can be approximated to an elliptical helix. To this end, we performed a k-means clustering over the ensemble of such helices on the three parameters, (1) major axis, (2) minor axis of the ellipse, and (3) pitch per turn of the helix, to group similar water wire geometries. Each water wire was represented as a point in a 3-dimensional space with coordinates equal to the magnitude of the three parameters respectively. K clusters were generated by assigning each data point to a cluster whose center is nearest to the data point. K is incremented over successive iterations and the highest intra-cluster variance is stored for every k. In factor analysis, scree plot² is a graphical representation of intra-cluster variance against a series of sequential cluster levels, where an appropriate number of clusters is defined as one at which the reduction of the variance slows significantly. Next, we used a scree plot to identify the optimal number of clusters (k) for which the maximum intra-cluster variance in minimum. We identified four unique water wire geometries through AQP1 (k^{optimal}=4) represented by four clusters (FIG. 10). Table 2 shows the parametric equation of the four characteristic water wires from four clusters. One water wire from each of the four clusters reveal the possible geometries water may take during pressure driven flow through the AQP1 channel. We selected the water wire closest to each cluster center as the “representative” wire for that geometry. Using the IPRO input language terminology in PoreDesigner, the protein molecule permitted to mutate is referred to as design molecule (DM) and the molecule they bind is referred to as target molecule (TM). Within the DM, the exact residues that are allowed to mutate are called design positions (DPs). In PoreDesigner, we declared the four water wires as target molecules (TMs) while the OmpF (2omf.pdb) and 25 of its pore constricting residues were defined as design molecules (DM) and design positions (DPs) respectively. We ran PoreDesigner for all four water wires (from k-means clustering) to identify convergent designs which are independent of the permeating water wire geometry.

TABLE 2

| Water wire | Parametric equation of representative |
|---|---|
| cluster no. | water wires from each cluster |
| 1 | x = 0.401 sinθ, y = 0.121 cosθ, z = 2.31 θ |
| 2 | x = 0.321 sinθ, y = 0.281 cosθ, z = 3.24 θ |
| 3 | x = 0.314 sinθ, y = 0.309 cosθ, z = 1.81 θ |
| 4 | x = 0.319 sinθ, y = 0.277 cosθ, z = 2.35 θ |

Preparatory Phase to k-Means Cluster 1,000,000 Water Wires

In order to discern the principal geometric modes of water permeation through AQP1, each water wire was fit to an elliptic helix equation. The parametric form of an elliptical helix is: x=R₁ cos θ; y=R₂ sin θ; z=Cθ.

A k-means clustering was performed on the semi major (R₁), semi minor (R₂) axes, and the pitch per turn (C) for one million generated elliptical helices to identify the different geometries of the water wire.

### Aligning OmpF Structure and Placing Water Wires

The following sub-steps were performed to prepare the OmpF molecule for redesign.

Step 1: The OmpF (2omf.pdb) structure was oriented such that the pore axis coincides with the Z-axis.
Step 2: The OmpF molecule was translated along the Z-axis to place the origin at the pore constriction center.
Step 3: The four water wires were placed one at a time in the channel cavity of OmpF such that the vertical axis of symmetry of the water wire helix coincides with the Z-axis (pore axis).
Step 4: The OmpF molecule was used as the design molecule (DM) and the water wire as the target molecule (TM) and the 25 pore constricting residues were defined as design positions (DPs) in the IPRO input file used in PoreDesigner. In IPRO DMs are the protein(s) that (may) undergo mutations, TMs are the molecule(s) that bind to the DMs and DPs are the list of residue positions in DPs that are allowed to mutate.

### OmpF Redesign Phase

In this phase, the OmpF DPs are allowed to mutate to create a narrow yet hydrophobic pore such that the resultant pore allows a single-file water transport at enhanced hydraulic permeation rates. A set of nine amino acids were chosen (Trp, Phe, Tyr, Ile, Met, Leu, Pro, Val, and Ala) as allowed redesign choices. A minimum of 50% of the newly introduced residues had to be either Trp, Phe or Tyr. This sped up the identification of acceptable designs as PoreDesigner would not have to “sift” through all possible combinations of smaller allowed amino acids (with low interaction energy with the water wire) which would ultimately be rejected as they did not meet the pore size cutoffs.

### PoreDesigner Iterative Redesign Cycle

The PoreDesigner iterative redesign cycle largely follows the sequence of steps defined in IPRO.

Step 1: Backbone perturbation of an 11-amino acid window with a randomly chosen DP as the sixth (central) amino acid is performed. The side chains are stripped, and backbone phi and psi dihedral angles are randomly perturbed using values from a normal distribution (μ=0,) σ=1.5°.
Step 2: Repacking the amino acids side chains inside and within 4.5 Å of the perturbed region and redesign of all DPs included within the perturbed region to any of the allowed amino acids is done. This optimization step is carried out by solving an MILP problem with an objective function of maximizing the interaction energy (van der Waals, electrostatics, and solvation). Constraints in the MILP formulation impose selection of only one amino acid rotamer at each design position, mutation of at least 50% of the DPs to longer side chain amino acids (Trp, Phe, Tyr, and Met), and prevention of the same amino acid-rotamer combination to be chosen at the same design position in follow up iterations.

This MILP formulation that is solved in Step 2 is stated as follows:

### Sets:

i,j=1, . . . , N, set of all design positions
r,s=1, . . . , R, set of rotamers for position i.
U={(i,r)|i=1, . . . , N;r=1, . . . , R} universal set of all feasible residue position and amino acid rotamer combinations.
CUTS={(i,r)|yᵢᵣ=1} set of residue position and amino acid rotamer combinations for the design obtained from any iteration
A={Trp, Tyr, Phe, Met, Ile, Leu, Val, Pro, Ala} set of allowed amino acids
U_{AA}=set of all amino acids

### Parameters:

ECᵢᵣ stores the interaction energy of rotamer r at position i and the non-rotamer region
ERᵢᵣ^{js} stores interaction energy between rotamer r at position i and rotamer s at position j

$${LONG}_{r} = \{ \begin{matrix}1 , & \begin{matrix}if r is a rotamer of a long side chain \\ amino acid ( Trp , Phe , Tyr , Met )\end{matrix} \\ 0 , & otherwise\end{matrix} \\ {SHORT}_{r} = \{ \begin{matrix}1 , & \begin{matrix}if r is a rotamer of a short side chain \\ amino acid ( Ile , Leu , Val , Pro , Ala )\end{matrix} \\ 0 , & otherwise\end{matrix} \\ M = maximum number of mutations allowed at each iteration . \\ {WT}_{ir} = \{ \begin{matrix}1 , & \begin{matrix}i f rotamer r is seen at design \\ position i in the wild type structure\end{matrix} \\ 0 , & otherwise\end{matrix}$$

### Binary Variables:

$${y}_{ir} = \{ \begin{matrix}1 , & if rotamer r is selected at position i \\ 0 , & otherwise\end{matrix} \\ {w}_{ir}^{js} = \{ \begin{matrix}1 , & if rotamer r is selected at position i and s at position j \\ 0 , & otherwise\end{matrix} \\ {z}_{ir} = \{ \begin{matrix}1 , & \begin{matrix}if rotamer r is selected with amino acid from \\ set A at position i upon mutation\end{matrix} \\ 0 , & if position i is unmutated\end{matrix}$$

### MILP Formulation

$$Maximize {\sum }_{i = 1}^{N} {\sum }_{r = 1}^{{R}_{i}} {y}_{ir} {EC}_{ir} + {\sum }_{i = 1}^{N - 1} {\sum }_{r = 1}^{{R}_{i}} {\sum }_{j = i + 1}^{N} {\sum }_{s = 1}^{{R}_{j}} {w}_{ir}^{js} {ER}_{ir}^{js}$$

subject to:

$$\begin{matrix} {\sum }_{r = 1}^{R} {y}_{ir} = 1 , ∀ i \vert 1 , … , N & ( 1 ) \\ {y}_{ir} = {\sum }_{s = 1}^{R} {w}_{ir}^{js} , ∀ i \vert 1 , … , N - 1 , ∀ j \vert i + 1 , … , N , ∀ r \vert 1 , … , R & ( 2 ) \\ {y}_{js} = {\sum }_{r = 1}^{R} {w}_{ir}^{js} , ∀ i \vert 1 , … , N - 1 , ∀ j \vert i + 1 , … , N , ∀ s \vert 1 , … , R & ( 3 ) \\ {\sum }_{i = 1}^{N} {y}_{ir} {LONG}_{r} - {\sum }_{i = 1}^{N} {y}_{ir} {SHORT}_{r} \geq 0 , ∀ r ∈ A & ( 4 ) \\ {\sum }_{r = 1}^{R} {\sum }_{i = 1}^{N} {z}_{ir} \leq M & ( 5 ) \\ {\sum }_{r = 1}^{R} {z}_{ir} \leq 1 , ∀ i \vert 1 , … , N & ( 6 ) \\ {z}_{ir} = 0 , ∀ i \vert 1 , … , N , ∀ r ∈ {U}_{AA} \backslash A & ( 7 ) \\ {y}_{ir} \geq {z}_{ir} , ∀ i \vert 1 , … , N , ∀ r \vert 1 , … , R & ( 8 ) \\ {y}_{ir} \geq ( 1 - {\sum }_{r = 1}^{R} {z}_{ir} ) {WT}_{ir} & ( 9 ) \\ {\sum }_{{r = 1}_{( i , r ) ∈ CUTS}}^{R} {\sum }_{i = 1}^{N} {y}_{ir} + {\sum }_{{r = 1}_{( i , r ) ∈ U \backslash CUTS}}^{R} {\sum }_{i = 1}^{N} ( 1 - {y}_{ir} ) \leq ( R \times N ) - 1 & ( 10 )\end{matrix}$$

The objective function maximizes the net interaction energy of the rotamers with the non-rotamer portion of the binding assembly and with each other. Constraint 1 ensures that exactly one rotamer is selected at each design position. Constraints 2 and 3 ensure that wᵢᵣ^{js} is one only when both yᵢᵣ and yⱼₛ have a value of one. Constraint 4 ensures that at least 50% of the DPs are mutated to longer side chain residues (Trp, Phe, Tyr, and Met). This alleviates the need to cycle through designs with all smaller side chain residues (Ile, Leu, Val, Pro, and Ala) and this accelerates convergence. Using a very low percentage will result in longer run times owing to small side chain-rich designs being identified first which will be weaned out at the pore size check at step 5. On the other hand, a very high percentage will result in steric clashes between the chosen long side chain residues yielding larger than expected pore sizes (akin to OCDTFTrp design). Constraint 5 ascertains that at most M out of N design positions are allowed to mutate in a given iteration. The value of M is randomly generated for each PoreDesigner iteration and fed as a parameter to the MILP step. Constraint 6 makes sure that if a design position is to be mutated, it assumes only one new rotamer and if unmutated it retain the wild type amino acid rotamer while constraint 7 prevents mutation to non-hydrophobic residues. Constraints 8 and 9 together pass on the information about the current design to the objective function using the binary variable yᵢᵣ. If a design position is mutated, constraint 8 uses zᵢᵣ to set the yᵢᵣ value for that position and rotamer combination to one. However, if a design position is not mutated (i.e. zᵢᵣ=0), constraint 9 uses parameter WTᵢᵣ to set the yᵢᵣ value to one corresponding to the wild-type configuration of that residue. Therefore, an MILP design has yᵢᵣ values of one for each design position obtained either from a mutation or from the wild type configuration (if unmutated). At the end of each iteration the design is appended to a CUTS set. Constraint 10 makes sure none of the existing designs from the CUTS set are chosen in the current iteration.

Step 3: A local, rigid-body docking of the water wire using random translations in the X, Y, and Z directions by sampling coordinates for the water wire from a normal distribution centered at zero and standard deviations of 0.2 Å, 0.2 Å, and 2 Å, respectively.
Step 4: A force field complex energy minimization in Cartesian coordinates x,y,z using a gradient based search.
Step 5: Pore size analysis is performed using a PoreAnalyzer module to check if it satisfies the desired pore size. The interaction energy is calculated if the pore opening is within the desired size range, otherwise the design is discarded.
Step 6: If the design is accepted in the previous step, the interaction energy between the water wire and the redesigned OmpF is calculated. Redesigns with lower interaction energy than the currently best are always accepted. Redesigns with larger interaction energies (in absolute magnitude) are accepted with a probability or a Boltzmann factor equal to

$${e}^{\frac{- Δ ( interaction energy )}{kT}}$$

(i.e., Metropolis criterion where, k is the Boltzmann constant and is ˜0.33×10⁻²³ cal/K, and T is the temperature in K). A temperature of 3,640 K in the Boltzmann factor is used which ensures that there is a 25% probability that a redesign with an interaction energy 10 kcal/mol more negative than the best so far will be retained.
Step 7: A cumulative set of integer cuts which stores information about the current mutation (residue, position, rotamer). This ensures that no redesign (either accepted or rejected previously) is revisited.
Step 8: A number of perturbation/redesign iterations of PoreDesigner are performed until a pre-specified number of accepted redesigns in terms of pore size are retrieved (i.e., typically we require 30 accepted redesigns).

### Post-Redesign Analysis of Results

This step is used to estimate the pore constriction diameter of the designed OmpF mutant and accept or discard designs accordingly.

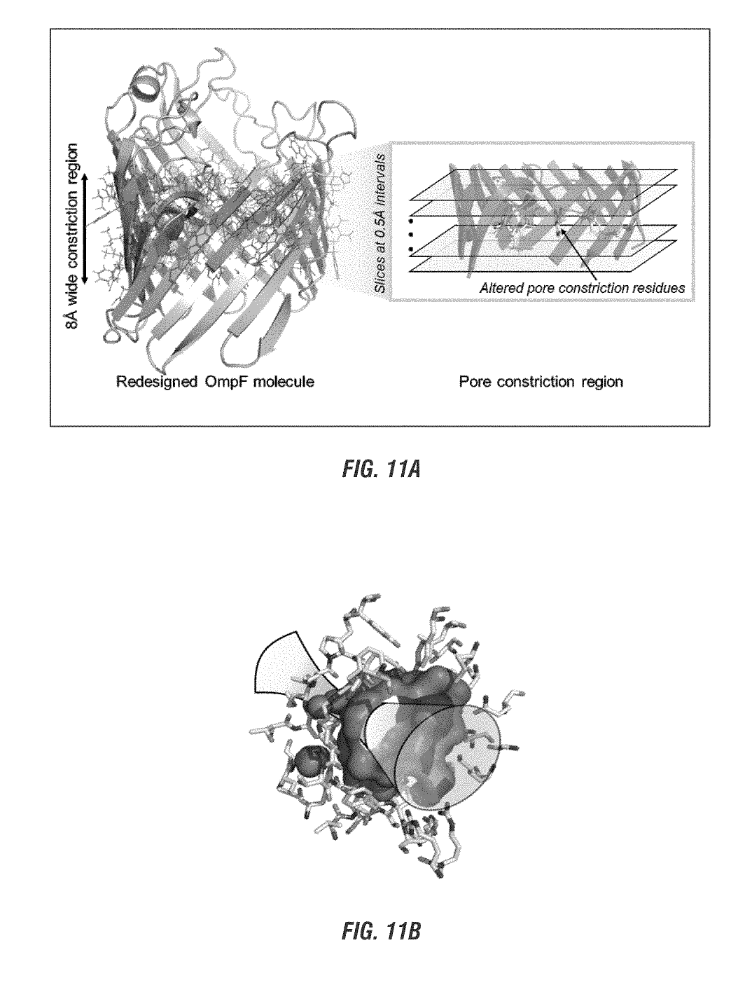

Step 1: Introduce in the ˜8 Å long constriction region (FIG. 11) perpendicular planes at every 0.5 Å (approximately 16 slices).
Step 2: Apply developed PoreAnalyzer algorithm comprised of the following sub-steps:
1. Supply the oriented OmpF pdb structure.
2. Identify the list of pore center coordinates at each of the 16 slices (FIG. 11) of the pore constriction region.
3. At each slice, find the coordinates of the pore constricting atoms (including their van der Waals radii) nearest to the pore center coordinates.
4. At each slice, fit the largest ellipse (Ellipse fitting method for details) that just touches the pore constricting atoms (with no atoms inside the ellipse). The ellipse is centered at the pore center coordinates for a given slice.
5. Store the major axis dimensions (D1) of the ellipses from each slice in a list D^{pore} such that, Dᵢ ∈ D^{pore}. The complete set of major axes for the ˜8 Å constriction region is stored in D^{pore}.
6. The minimum value in D^{pore} (i.e. ᵢMin D^{pore}=Dₘᵢₙ^{pore} determines the actual pore bottleneck diameter or the pore constriction diameter (FIG. 11).
7. The minor axis dimension corresponding to the ellipse for which major axis is equal to Dₘᵢₙ^{pore} is identified and thus the pore constriction dimensions are determined.
   Step 3: Impose a design check to retain OmpF redesigns if it meets the desired pore size criteria. The first check is imposed while designing AQP-like small pores (<4 Å diameter) that allow single file water transport. The latter is used for redesigning OmpF for selective separation (rejects aqueous solute A but not B) of aqueous solutes (A and B) with hydrodynamic diameters D_{A} and D_{B} respectively.
   Check 1: Accept the design if Dₘᵢₙ^{pore}<4 Å is satisfied.
   Check 2: Accept the design if both Dₘᵢₙ^{pore}<D_{A} and Dₘᵢₙ^{pore}>D_{B} are satisfied.

Accepted OmpF designs are sorted in decreasing order of the DM-TM interaction energy implying that redesigns with least interaction with the water wire are ranked higher. Thus, PoreDesigner can be used to create the selective internal structure of AQP1 (or any desired pore size) inside the stable beta-scaffold of OmpF.

Ultimately, we obtain the structures for the final 100 frames of MD simulation of pressure driven water transport through OmpF and the mutant designs. We use the PoreAnalyzer module on them to report the predicted pore sizes. This is to ensure we track any pore widening that might occur during water permeation.

### Ellipse Fitting Method

At each slice of pore contriction region we fit the general equation of a rotated ellipse Ax²+Bxy+Cy²+Dx+Ey+F=0 using non-linear regression. Here, the coefficients A, B, C, D, E, and F represent arbitrary real-valued constants with at least one of A, B, or C as well as at least one of D, E, or F nonzero. Although all conic sections can be represented in this way, some combination of the constants could give rise to one of the five degenerate conic sections (a point, a line, or two intersecting lines, two parallel lines or the empty set). But we verified that resulting figure is a non-degenerate conic section (circle or ellipse) by checking that the area inside the curve after the non-linear regression was less than 60. This is because the wild type OmpF pore area with major and minor axes lengths 11 Å and 7 Å respectively, is 60.47 Å².

### Inner, Outer Pore Wall and Overall Hydrophobicity Scores

We used the ΔG_{transfer}^{water→ethanol} values (from the Kyte-Doolittle (KD) hydrophobicity scale) to evaluate the inner pore wall, outer pore wall and overall KD-hydrophobicities of the OCD-TFTrp, UCD, and CSD designs which were subsequently purified and expressed, and embedded in vesicles for transport experiments. KD-hydrophobicities have been used as a standard to assess the performance of novel hydrophobicity scales and the accuracy of the KD scale in estimating protein hydrophobicities is considerably reliable. Each of the three hydrophobicity scores were calculated by adding the products of amino acid (i) frequencies (nᵢ) to their individual ΔG_{transfer}^{water→ethanol} values. Amino acid frequencies refer to the number of times (nᵢ) a single amino acidi occurs. The overall hydrophobicity score of a channel protein is the sum of its inner pore wall and outer pore wall hydrophobicity scores calculated from transfer free energies (equation 11).

$${\sum }_{i = 1}^{20} ( {n}_{i}^{inner \_ porewall} \times Δ {G}_{{transfer}_{i}}^{water -> ethanol} ) + {\sum }_{i = 1}^{20} ( {n}_{i}^{outer \_ porewall} \times Δ {G}_{{transfer}_{i}}^{water -> ethanol} ) = Overall hydrophobicity score \qquad ( 11 )$$

In order to decide if a given amino acid is a part of the inner pore wall or the outer pore wall, Python 2.7 scripts were written. If the distance between the pore center and the Cα atom of any amino acid is greater than that from its Cβ atom (or analogous atom), then it is counted as an inner pore wall residue. Otherwise, it is counted an outer pore wall residue.

### Molecular Dynamics Methods

All MD simulations were performed using the program NAMD, a 2 fs integration time step, and 2-2-6 multiple time-stepping. Parameters for the POPC lipid-bilayer, OmpF protein, and ions were taken from the CHARMM36 parameter set with the CMAP corrections. A TIP3P model was used for water. All simulations employed a 10-12 Å cutoff for van der Waals and short-range electrostatic forces, the particle mesh Ewald (PME) method for long-range electrostatics computed over a 1.1 Å grid and periodic boundary conditions. Simulations in the NPT (constant number of particles N, pressure P, temperature T) ensemble were performed using a Lowe-Andersen thermostat and Nosé-Hoover Langevin piston pressure control set at 295 K and 1 atm, respectively. Visualization and analysis were performed using VMD.

The wild type OmpF protein was obtained from protein database with the accession code, 2OMF. Mutant OmpF proteins were obtained from the PoreDesigner algorithm. The POPC lipid bilayer was built using the Membrane Builder plugin in VMD. An OmpF timer was subsequently inserted into the lipid membrane and any overlapping lipids were deleted or removed to maintain the area per lipid at a constant value (70 Å²). Two sets of systems were created for the wild type and each OmpF mutants; the first set had a solution of Na⁺ ions to generate an electrically neutral system and a second set contained a solution of 1M NaCl. Thereafter a total of eight systems were constructed, each measuring 14.3 nm×14.3 nm×7.2 nm. A periodic boundary was employed along the xy-plane and the system comprised 111,500 atoms.

After assembly, each system was energy minimized for 5000 steps and equilibrated for 45 ns in the NPT ensemble. To induce an ionic current, systems containing 1M NaCl were simulated for an additional 35 ns in the presence of a uniform electric field. A 500 mV voltage bias produced by the field follows V=−L_{z}E_{z}, where L_{z} is the length of the unit cell along the z-axis. All simulations in the presence of an electric field (˜0.1538 kcal/(mol Å e)) were simulated in an NVT (constant number of particles N, volume V, temperature T) ensemble.

Calculation of the osmotic permeability utilized a collective diffusion model. Permeability analysis was performed in the last 30 ns of the 35 ns equilibrium trajectories of systems in solutions containing only Na⁺ ions. To this end, diffusion coefficient, D, of water molecules moving through each channel was computed before estimating the osmotic permeability as P_{f}=v_{W}×D where, v_{W} is the volume of a single water molecule.

Ultimately, the conductance through the channels were calculated using standardized methods measuring the displacement of ions through the protein channel during the last 25 ns of the 35 ns simulations of the systems subject to an applied external electric field.

### Example 3: Estimation of Maximum OmpF Conductances During Salt Removal

A simple theoretical analysis of ion conductance targets for desalination membranes based on OmpF channels is described in this Example. In this calculation, we used wild type OmpF as an example to discuss based on the single channel (protein) permeability and simulated protein conductance, what conductance value would lead to drinking water quality for a seawater feed and then we extend this analogy to find similar results for other feed types. The overall approach is to assume the thermodynamic equivalency of the electrochemical driving force for which conductance measurement was obtained to the concentration driven driving force that would drive ions across the membrane. In summary, from this approximate analysis, we find that a conductance as low as ≤0.018 nS would be needed for seawater desalination (assuming a feed of 35 g/L), ≤0.034 nS for brackish water desalination (assuming a feed of 5 g/L), and ≤0.056 nS for treating low salinity wastewater (assuming a feed salinity of 2 g/L). All these targets can be possibly met by the UCD and OCD designs as their simulated conductances lie within the range simulated for these mutants. An example calculation is described in detail below.

For the seawater case, the salinity (NaCl mass concentration) on the feed side is 35 g/L while the salinity (NaCl mass concentration) on the permeate side is 0.5 g/L (which is the salinity of drinkable water). Due to the concentration difference between feed side and the permeate side, there will be NaCl flux across the membrane, defined as the flux J_{NaCl}. We also assume that the molar ratio of Na+ and Cl− ions in the feed is approximately 1:1.

Now, we consider a situation that applying an electric field on the opposite direction (which means there is high voltage on the permeate side while low voltage on the feed side) to create driving force thermodynamically equivalent to the expected solute (Na⁺) passage under a concentration gradient condition. A similar hypothetical analysis could also be conducted for Cl⁻ in the opposite direction.

Based on the Nernst-Planck equation:

$${J}_{net} = {J}_{Na} + {J}_{ele} = - D \frac{dC}{dx} - uCzF \frac{dV}{dx} \qquad ( 12 )$$

where D is the diffusion coefficient of the solute, u is the mobility of an ion, and D=uRT based on Einstein relation.

### Thus,

$${J}_{net} = {J}_{Na} + {J}_{ele} = - uRT \frac{dC}{dx} - uCzF \frac{dV}{dx} \qquad ( 13 )$$

when the net flux Jₙₑₜ is zero, J_{Na}=−Jₑₗₑ:

$$\begin{matrix}\frac{dV}{dx} = - \frac{RT}{zF} \frac{d ln C}{dx} & ( 14 ) \\ Δ {V}^{*} = {V}_{feed} - {V}_{permeate} = - \frac{RT}{zF} ln \frac{{C}_{feed}}{{C}_{permeate}} & ( 15 )\end{matrix}$$

Based on the Na⁺ conductance results from simulation, we calculated the ion (Na⁺) transport rate:

$${I}^{*} = V * G = ( - \frac{RT}{zF} ln \frac{{C}_{feed}}{{C}_{permeate}} ) * G \qquad ( 16 )$$

Under this condition, it means when the voltage difference between feed side to permeate side is ΔV* on the membrane model above, there will be no Na⁺ flux across the membrane, since the feed side and permeate side reach chemical potential equilibrium. And based on the conductance simulation results, the ion (Na⁺) transport rate that provides the 54 mV voltage difference is I*. Thus, when there is no electric field applied across the membrane, Jₙₑₜ is maximum, and the Na⁺ flux equals to the ion transport rate due to a ΔV* voltage difference. So the maximum ion transport rate is I* when the salinity on the feed side is 35 g/L while the salinity on the permeate side is 0.5 g/L (assuming an equimolar mixture of Na⁺ and Cl⁻). Based on the single OmpF (wild type) permeability p_{f} that's measured in this paper, so when there is no voltage applied, the molar ratio of water transport rate to NaCl transport rate is

$$\frac{pf}{{I}^{*}} ,$$

which equals to 1.94% (w/w) Na⁺ the product solution. Thus, on the permeate side, we will end up with 1.94 g Na⁺/L water when operating a OmpF (wild type) based membrane, compared to 21.2 g/L Na⁺ on the feed side.

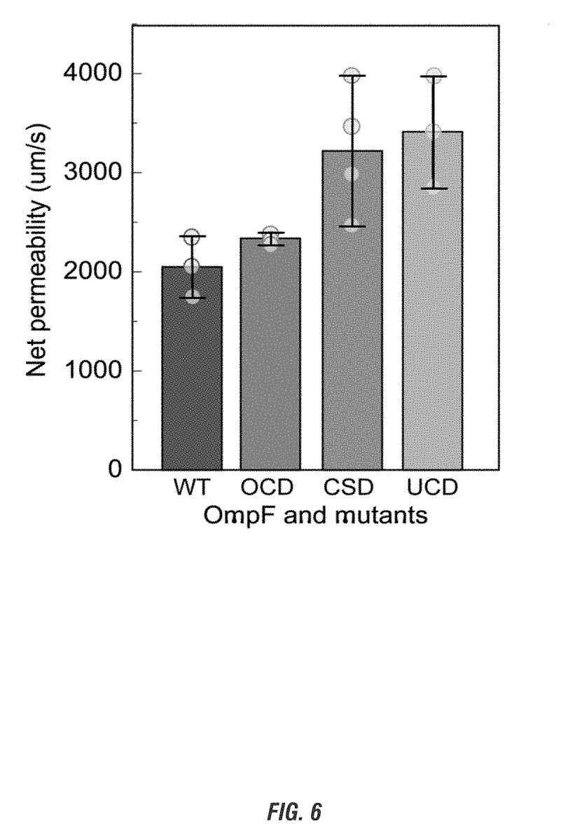

Using the above analysis, we can calculate at what maximum Na⁺ conductance value, we may expect to see sufficient salt rejection for seawater desalination leading to a sodium concentration equivalent to a 0.5 g/L NaCl permeate by simply scaling the WT conductance values that provides the above concentration to the desired Na+ value in permeate (˜0.303 g/L of Na+). Table 3 shows the maximum calculated conductance values for achieving drinking water quality values in permeate from hypothetical OmpF mutant membranes. We can do a similar analysis to find the maximum Cl⁻ conductance value that would lead to the 0.5 g/L NaCl product for chloride. Upon adding the two values we find that a value of 0.018 nS/cm would be sufficient if the WT water permeability (the lowest OmpF measured among the mutants) is assumed. This conductance value is within the range of values estimated using simulations for the UCD and OCD mutants and thus we could assume that both the UCD and OCD mutants would be expected to be a key transport element of a successful OmpF mutant based biomimetic desalination membrane if synthetic hurdles are overcome. Net permeability of OmpF and its mutants in reconstituted biomimetic membranes are shown in FIG. 6.

TABLE 3

| | | Na⁺ conductance | | Cl⁻ conductance | |
|---|---|---|---|---|---|
| | | (0.6 nS) | | (0.26 nS) | |
| | | Final Na+ | | Final Cl− | |
| | | conc of | | conc of | |
| Water | Salinity | permeate | Ideal Na⁺ | permeate | Ideal Cl⁻ |
| type | (g/L) | (g/L | conductance | (g/L) | conductance |
| Seawater | 35 | 19.4 | 0.009 | 5.5 | 0.009 |
| Brackish | 5 | 10.6 | 0.017 | 3 | 0.017 |
| water | | | | | |
| Waste | 2 | 6.4 | 0.028 | 1.8 | 0.028 |
| water | | | | | |

This analysis reveals that in order for such membranes to be usable for seawater desalination, the estimated maximum total conductance values is 0.018 nS for seawater desalination (assuming a feed of 35 g/L NaCl). Similarly, for brackish water desalination this maximum is 0.034 nS (assuming a feed of 5 g/L NaCl), and 0.056 nS for low salinity wastewater (assuming a feed salinity of 2 g/L NaCl). All these conductance target values can be met by the derived UCD and OCD designs as their simulated conductance values are in the requisite range.

### Example 4: Membrane Compatibility Enhancement

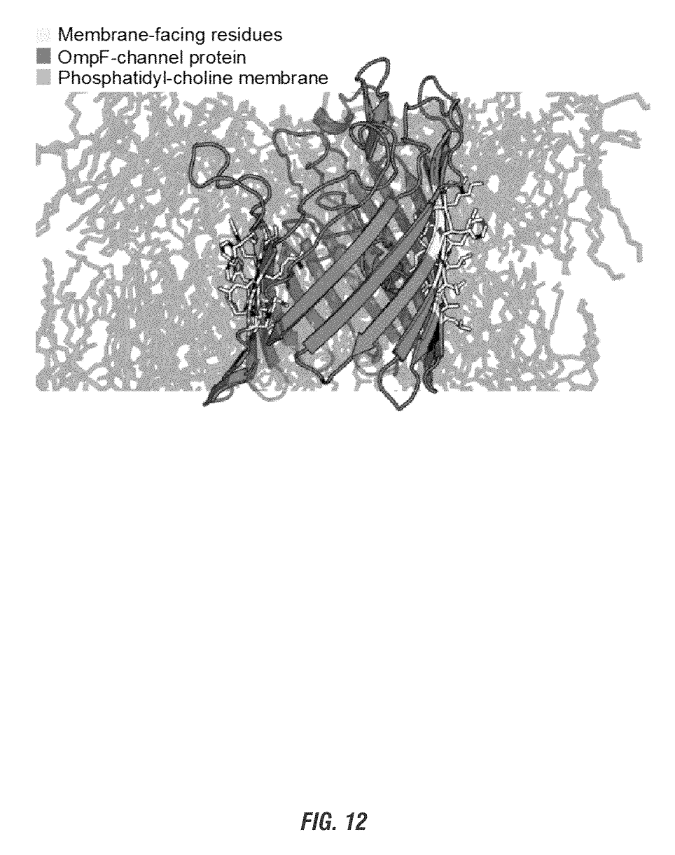

We use PoreDesigner to systematically alter the membrane-facing residues (amino acids/functional groups) to make the channel protein compatible with various membrane materials. We identify the membrane-facing amino acids on the channel protein (OmpF) (FIG. 12) and feed that to the PoreDesigner algorithm as input. Depending on the membrane material (phosphatidyl-choline, synthetic membrane, etc.), PoreDesigner suggests alternative amino acids on the pore surface that ensure favorable thermodynamic interactions at a molecular level and result in stable membrane-protein filtration assemblies. We then scale make viable 2D-membrane sheets for high-throughput bioseparations using these biomimetic membranes. Applications range from desalination (separating salt from water to harness marginal water resources), extracting pure water from urine which can be used in international space stations, and separating solutes with sizes not differing by more than half an angstrom. We have already demonstrated a proof-of-concept by performing perfect desalination and separation of glucose from sucrose using these pores.

### Example 5: Rejection of Protons for Non-Invasive MRI

We engineer hydrophilic residues at the pore constriction to ensure that we can reject protons through the channels which bears applications in non-invasive MRI scanning methods.

Injecting customized bacterial cells into the bloodstream of a patient can be used to (a) target them to reach a particular organ of interest and (b) capture high-resolution images of that organ. The technique involves overexpressing water channel proteins on these bacterial cell membranes such that they can intake and in turn release more water across their membranes to maintain their own osmotic pressures. Now, the diffusion-weighted MRI technique uses sound waves to capture the vibration of these water molecules ejected from these bacterial cells after they have been targeted to reach a particular organ of a patient (such as the liver). The faster these water molecules are being ejected from the bacterial cells, the higher is the resolution of the image obtained from the patient.

Current techniques use bacterial transmembrane water channel protein because the wild type outer-membrane porin (OmpF) has a bigger pore size which allows amino acids and sugars through them, which affects the final resolution of the image. Therefore, we use PoreDesigner-designed custom OmpF channels that not only reject protons but also permeate water at an order of magnitude higher rate (as shown in Example 1) than any aquaporin ever reported.

The importance of rejecting protons amounts to the fact that an infinite proton influx reduces all chemical moieties inside the cell and would result in death of these bacterial cells thus preventing them from being used for the intended purpose. In order to reject protons, we are taking inspiration from the aquaporin proton rejection mechanism.

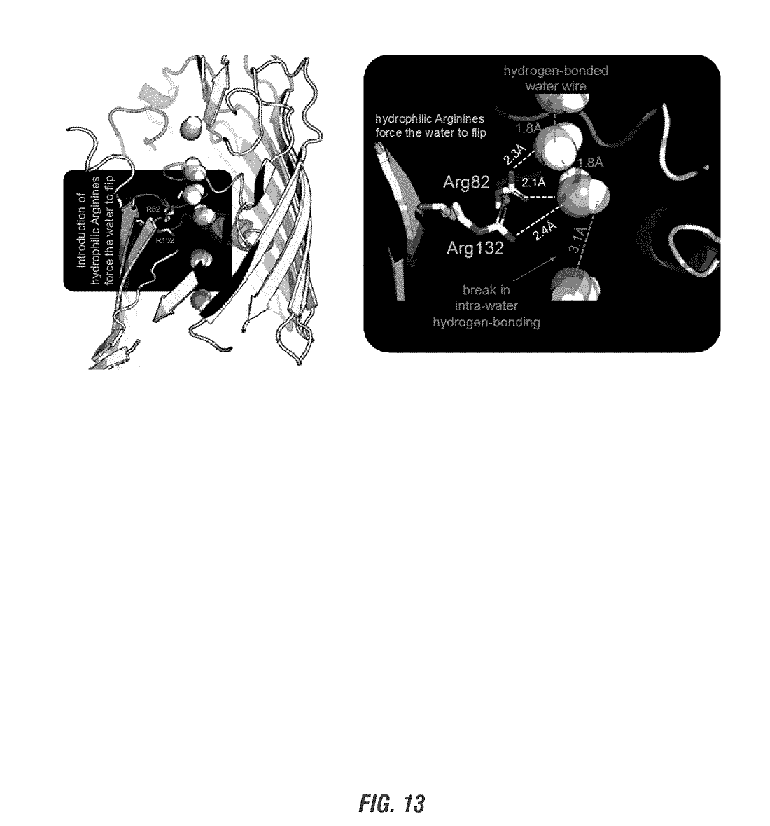

When water permeates through aquaporin as a molecule-wide water stream, near the constriction region, a polar amino acid (asparagine) pulls the oxygen of the water towards itself thus breaking the water stream. Beyond this point, downstream through the channel, the water molecules appear rotated by a complete 180 degrees (FIG. 13). This prevents the protons from jumping from one water molecule to the next (via intermediate hydronium ion formation) due to a larger jump length. A similar hydrophilic domain (using Arg82 and Arg132—as shown in FIG. 13) is used to achieve complete rejection of protons through our redesigned OmpF. As discussed in Example 1, the narrowest OmpF redesign we synthesized limits the passage of any solute bigger than water. Modification of this OmpF to reject protons render it a very strong candidate for non-invasive MRI.

### Example 6: Engineering DNA Translocation Channels for Nucleotide Sequencing

We use PoreDesigner to engineer the pore mouth of a DNA-translocation channel protein (CsgG) to employ it for DNA sequencing applications. We alter the hydrophobicity of the pore mouth of the CsgG protein to obtain a collection of CsgG mutants (redesigns) with low, medium, and high hydrophobicities. Lysine, arginine, and cystine are low hydrophobicity amino acids, leucine, isoleucine, and valines are medium, and phenylalanine and tryptophans are highly hydrophobic residues.

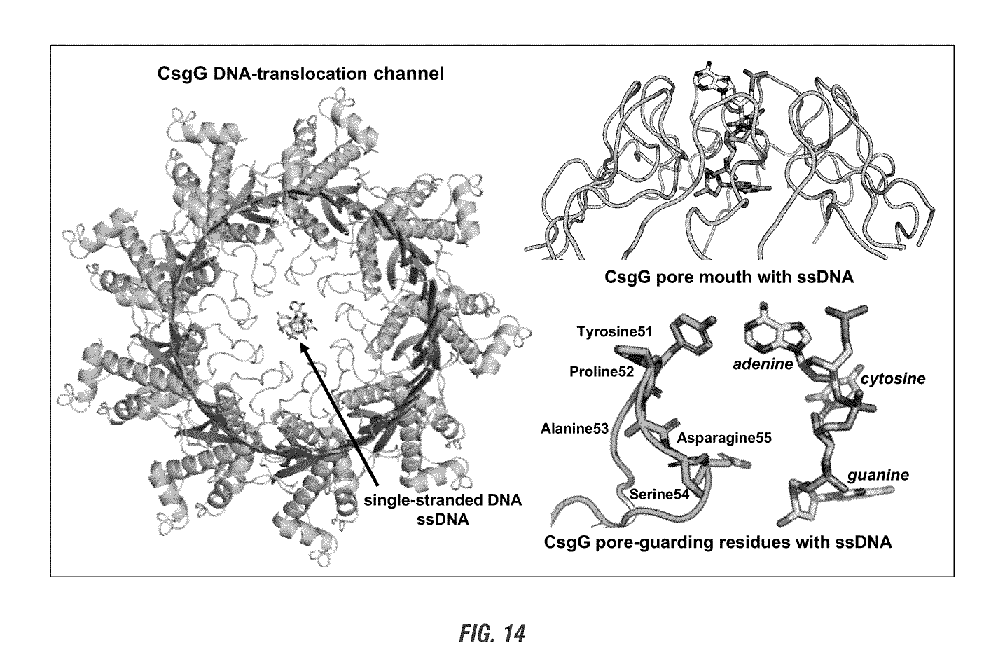

The experimental set up has the nanopore (CsgG) embedded in a synthetic membrane, which separates two buffer filled compartments (usually KCl-based buffers). A positive charge is applied to one compartment, resulting in ions from the solution forming a current that flows through the pore that can be measured. In the other compartment single strands of DNA associated with a molecular motor protein are attracted to this positive potential on the other side of the membrane and are coerced to translocate through the pore. The molecular motor protein controls the speed of the DNA as it goes through the pore. Within the CsgG pore there is a constriction point on the pore mouth (FIG. 14), meaning that when a single nucleotide sits in this constriction the reduction in the current will be specific to this base, allowing the sequence of the DNA to be determined.

However, measurements have revealed that using the CsgG nanopore as is results in poor resolution of the signals from all four types of nucleotides (adenine, thymine, cytosine, and guanine). To this end, PoreDesigner-generated designs with varying hydrophobicities at the pore mouth are tested to identify designs which show maximum resolution between signals from four nucleotide types. Altering the hydrophobicities yield varying molecular interactions between the pore wall and the nucleotides resulting in a sharp difference in the subsequent electrical signal.

>CsgG
(SEQ ID NO: 5)
CLTAPPKEAARPTLMPRAQSYKDLTHLPAPTGKIFVSVYNIQDETGQFKP
YPASNFSTAVPQSATAMLVTALKDSRWFIPLERQGLQNLLNERKIIRAAQ
ENGTVAINNRIPLQSLTAANIMVEGSIIGYESNVKSGGVGARYFGIGADT
QYQLDQIAVNLRVVNVSTGEILSSVNTSKTILSYEVQAGVFRFIDYQRLL
EGEVGYTSNEPVMLCLMSAIETGVIFLINDGIDRGLWDLQNKAERQNDIL
VKYRHMSVPPES

The inventions being thus described, it will be obvious that the same may be varied in many ways. Such variations are not to be regarded as a departure from the spirit and scope of the inventions and all such modifications are intended to be included within the scope of the following claims. The above specification provides a description of the manufacture and use of the disclosed compositions and methods. Since many embodiments can be made without departing from the spirit and scope of the invention, the invention resides in the claims. The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof

What is claimed is:

1. A biomimetic membrane comprising:
a mutant protein pore having an altered pore size and/or hydrophobicity.

2. The biomimetic membrane of claim 1, wherein the mutant protein pore has a pore size from about 3 Å to about 10 Å.

3. The biomimetic membrane of claim 1, wherein the mutant protein pore has an off-center pore closure design, a uniform pore closure design, or a cork-screw design.

4. The biomimetic membrane of claim 1, wherein the mutant protein pore comprises a substitution of one or more wild type pore constriction residues with a hydrophobic amino acid.

5. The biomimetic membrane of claim 1, wherein the mutant protein pore is a mutant β-barrel protein pore.

6. The biomimetic membrane of claim 5, wherein the mutant β-barrel protein pore is a porin.

7. The biomimetic membrane of claim 5, wherein the porin is Outer Membrane Protein F (OmpF).

8. The biomimetic membrane of claim 5, wherein said porin is CsgG.

9. The biomimetic membrane of claim 1, further comprising a lipid or a block copolymer.

10. A composition comprising the biomimetic membrane of claim 1, wherein the composition is for use in precision separations, air purification, water filtration, desalination, non-invasive MRI, or DNA sequencing.

11. A method of redesigning the pore size of a protein pore comprising:
selecting a desired pore size; and
altering one or more wild type pore constriction residues of the protein pore.

12. The method of claim 11, wherein the desired pore size is from about 3 Å to about 10 Å.

13. The method of claim 11, wherein the one or more wild-type pore constriction residues are substituted with a hydrophobic amino acid.

14. The method of claim 13, wherein the hydrophobic amino acid is tryptophan, phenylalanine, or tyrosine.

15. The method of claim 11, wherein the protein pore is a β-barrel protein pore.

16. The method of claim 15, wherein the β-barrel protein pore is OmpF.

17. The method of claim 11, wherein the altering results in an off-center pore closure design, a uniform pore closure design, or a cork-screw design.

18. The method of claim 11, further comprising:
altering one or more wild type membrane contacting residues of the protein pore.

19. The method of claim 11, further comprising:
incorporating the protein pore into a membrane.

20. The method of claim 19, wherein the membrane comprises a lipid or a block copolymer.


SEQUENCE LISTING (5 sequences)

SEQ ID NO: 1 - protein, 340 aa, Escherichia coli
AEIYNKDGNKVDLYGKAVGLHYFSKGNGENSYGGNGDMTYARLGFKGETQINSDLTGYGQWEYNFQGNNSEGADAQTGNKTRLAFAGLKYADVGSFDYGRNYGVVYDALGYTDMLPEFGGDTAYSDDFFVGRVGGVATYRNSNFFGLVDGLNFAVQYLGKNERDTARRSNGDGVGGSISYEYEGFGIVGAYGAADRTNLQEAQPLGNGKKAEQWATGLKYDANNIYLAANYGETRNATPITNKFTNTSGFANKTQDVLLVAQYQFDFGLRPSIAYTKSKAKDVEGIGDVDLVNYFEVGATYYFNKNMSTYVDYIINQIDSDNKLGVGSDDTVAVGIVYQF

SEQ ID NO: 2 - protein, 340 aa, Artificial Sequence
  Synthetic construct
AEIYNKDGNKVDLYGKAWGWHYFSKGNGENSYGGNGDWTWARLGFKGETQINSDLTGYGQWEYFFQGNNSEGADAQTGNKTWLAFAGLKYADVGSFDYGRNYGVVYDALWYTDMLPAFWGDTWYWDDFFVFRVGGVATYRNSNFFGLVDGLNFAVQYLGKNERDTARRSNGDGVGGSISYEYEGFGIVGAYGAADRTNLQEAQPLGNGKKAEQWATGLKYDANNIYLAANYGETRNATPITNKFTNTSGFANKTQDVLLVAQYQFDFGLRPSIAYTKSKAKDVEGIGDVDLVNYFEVGATYYFNKNMSTWVDYIINQIDSDNKLGVGSDDTVAVGIVYQF

SEQ ID NO: 3 - protein, 340 aa, Artificial Sequence
  Synthetic construct
AEIYNKDGNKVDLYGKAWGWHYFSKGNGENSYGGNGDWTWARLGFKGETQINSDLTGYGQWPYWFQGNNSEGADAQTGNKTWLAFAGLKYADVGSFDYGRNYGVVYDALWYTWMLPWGWGDTWYSDDFFVFRVGGVATYRNSNFFGLVDGLNFAVQYLGKNERDTARRSNGDGVGGSISYEYEGFGIVGAYGAADRTNLQEAQPLGNGKKAEQWATGLKYDANNIYLAANYGETRNATPITNKFTNTSGFANKTQDVLLVAQYQFDFGLRPSIAYTKSKAKDVEGIGDVDLVNYFEVGATYYFNKNMSTGVDYIINQIDSDNKLGVGSDDTVAVGIVYQF

SEQ ID NO: 4 - protein, 340 aa, Artificial Sequence
  Synthetic construct
AEIYNKDGNKVDLYGKAWGWHYFSKGNGENSYGGNGDWTWAWLGFKGETQINSDLTGYGQWWYWFQGNNSEGADAQTGNKTWWAFAGLKYADVGSFDYGRNYGVVYDAWWYTWWWPWWWWWTWWWDDFFWWWVGGVATYRNSNFFGLVDGLNFAVQYLGKNERDTARRSNGDGVGGSISYEYEGFGIVGAYGAADRTNLQEAQPLGNGKKAEQWATGLKYDANNIYLAANYGETRNATPITNKFTNTSGFANKTQDVLLVAQYQFDFGLRPSIAYTKSKAKDVEGIGDVDLVNYFEVGATYYFNKNMSTYVDYIINQIDSDNKLGVGSDDTVAVGIVYQF

SEQ ID NO: 5 - protein, 277 aa, Escherichia coli
MQRLFLLVAVMLLSGCLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES